(19) United States
(12) Patent Application Publication
(10) Pub. No.: US 2022/0177398 A1
(43) Pub. Date: Jun. 9, 2022

(54) Industrial Process for Manufacturing of Perfluoro (Methyl Vinyl Ether)(PFMVE) and of 2-Fluoro-1,2-Dichloro-Trifluoromethoxyethylene (FCTFE)

(71) Applicant: Fujian Yongjing Technology Co.,Ltd, Shaowu (CN)

(72) Inventors: WEIFEN LUO, Shaowu (CN); LVZHOU QIU, Shaowu (CN); RONGWEN DING, Shaowu (CN)

(73) Assignee: Fujian Yongjing Technology Co.,Ltd, Shaowu (CN)

(21) Appl. No.: 17/565,492

(22) Filed: Dec. 30, 2021

Related U.S. Application Data
(63) Continuation of application No. PCT/CN2021/120886, filed on Sep. 27, 2021.

(30) Foreign Application Priority Data
Nov. 6, 2020 (DE) 102020129325.9

(51) Int. Cl. C07C 41/06; C07C 41/42

(52) U.S. Cl. CPC C07C 41/06; C07C 41/42

(57) ABSTRACT

The invention relates to a new industrial process for manufacturing of perfluoro(methylvinylether) (PFMVE), and of 2-fluoro-1,2-dichloro-trifluoro-methoxyethylene (FCTFE), involving reactions in liquid phase and performing reactions in a microreactor. The invention also relates to a new industrial process for manufacturing of perfluoro(methyl vinyl ether) (PFMVE) by fluorination, i.e., perfluorination, of 2-fluoro-1,2-dichloro-trifluoromethoxy-ethylene (FCTFE) with HF (hydrogen fluoride) in the presence of a Lewis acid catalyst, again performing the reaction in liquid phase, and preferably in a microreactor.

### BACKGROUND OF THE INVENTION

### 1. Field of the Invention

The invention relates to a new industrial process for manufacturing of perfluoro(methylvinylether) (PFMVE), and of 2-fluoro-1,2-dichloro-trifluoro-methoxyethylene (FCTFE). The invention also relates to a new industrial process for manufacturing of perfluoro(methyl vinyl ether) (PFMVE) by fluorination, i.e., perfluorination, of 2-fluoro-1,2-dichloro-trifluoromethoxy-ethylene (FCTFE).

### 2. Description of the Prior Art

The compound perfluoro(methyl vinyl ether) (PFMVE), also named perfluoromethoxyethene (IUPAC) or perfluoromethoxyethylene, and the compound 2-fluoro-1,2-dichloro-trifluoromethoxyethylene (FCTFE), also named 2-fluoro-1,2-dichloro-trifluoromethyl-vinylether or 2-fluoro-1,2-dichloro-trifluoromethoxyethene (IUPAC), are known in the state of the art. They are halogenated derivatives of methoxyethene (H₃CO—CH═CH₂; CAS number: 107-25-5; other names are ethenyl methyl ether or vinyl methyl ether, but the preferred IUPAC name is methoxyethene), which in turn is a derivative of ethylene (IUPAC name: ethene; H₂C═CH₂; CAS number: 74-85-1).

Perfluoro(methyl vinyl ether), for example is a monomer used to make some fluoroelastomers.

The synthesis of these compounds, perfluoro(methyl vinyl ether) (PFMVE) and 2-fluoro-1,2-dichloro-trifluoromethoxyethylene (FCTFE), having the following formulae (I) and (II), is also known in the state of the art.

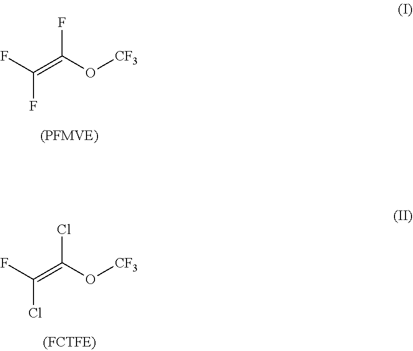 [SMILES: FC(F)=C(F)OC(F)(F)F ; F/C(Cl)=C(\Cl)OC(F)(F)F]

However, the known syntheses, as exemplified herein after, of the compounds perfluoro(methyl vinyl ether) (PFMVE) and 2-fluoro-1,2-dichloro-trifluoromethoxyethylene (FCTFE) have disadvantages, and there is a desire to provide improved processes of manufacturing the said compounds.

In early days, Du Pont in U.S. Pat. No. 3,180,895 (1965) disclosed a process for PFMVE out of reaction of hexafluoropropyleneoxide with acid fluorides followed by decarboxylation according to:

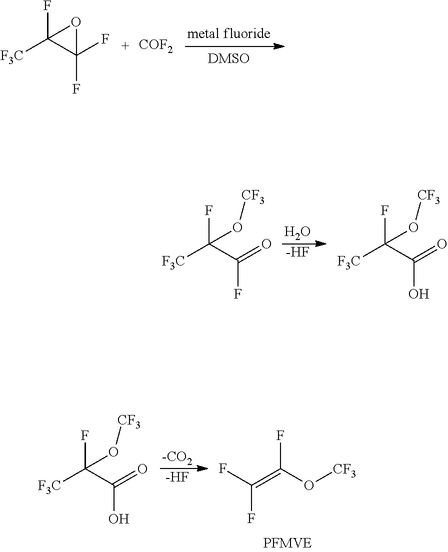

This route is quite complicated regarding handling, safety and availability of raw materials. Especially starting with toxic gaseous raw materials followed by liquid intermediates and intermediates in salt form (for decarboxylation usually the salt is preferred) which ends again in a gas is very challenging. Besides handling, lot of amounts of toxic waste water and toxic side materials are produced and causes environmental drawbacks.

A modification and improvement also described there already is the direct usage of the 2-perfluoromethoxy propionylfluoride over dry potassium sulphate pellets at 300° C. As this is no catalytic process the potassium sulphate cannot be recycled. Both procedures do not fit for large industrial scale.

Alternatively ZHONGLAN CHENGUANG CHEMICAL in CN1318366 (2005) disclosed a preparation of PFVME out of 1,2-dichloro-1,1,2-trifluoro-2-(trifluoromethoxy)ethane.

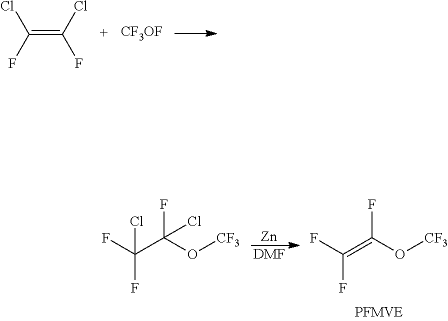

Another route is filed by SinochemLantian which contains the pyrolysis of the 2-perfluoromethoxy propionylfluoride in a fluidized bed in CN107814689 (2018). In another application, SinochemLantian in CN105367392 discloses the usage of CF3O-ammonium salt and it's reaction with chlorotrifluoroethylene but after reaction the work up is complicated, recycling of formed ammonium salts are not possible.

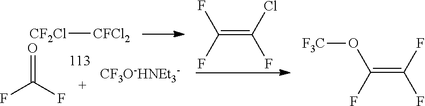

Known other methods for hydrogen containing derivatives are also quite complicated. For example, the trifluoromethoxy vinylether is disclosed in U.S. Pat. No. 3,162,622 from 1994. For this compound, which is technically much easier than the perfluoro(methylvinylether), Du Pont disclosed a process starting from halogeno-trifluoromethylvinylether by treatment with base. The starting material 2-chloro- or 2-bromo-trifluoromethyl-ether is prepared by a three step process starting with reaction of 2-halogenothanol and carbonyl fluoride to give an intermediate which is finally fluorinated to the 2-halogeno-trifluoromethyl-vinyl-ether with SF₄, here an example outlined for 2-chloroethanol:

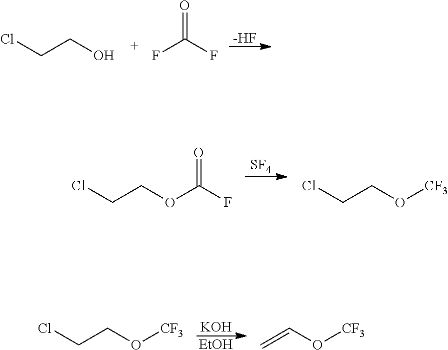

Other methods to trifluoromethoxy vinylethers are disclosed by Kamil et al. in Inorganic Chemistry (1986), 25(3), 376-80 where trifluoromethylhypochlorite is converted with halogenated olefins in a 1,2-additon reaction to the corresponding halogenated trifluoromethoxy halogenoalkane followed by H-Hal elimination:

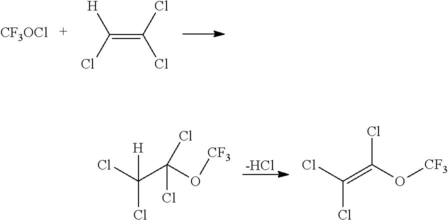

The CF₃OCl is known to be prepared by reaction of carbonyl fluoride and ClF like disclosed in DE1953144 (1969). Solvay Specialty Polymers in EP1801091 (2007) discloses the addition of CF3OF to Trichloroethylene in a stirred vessel and this same reaction but using a so called microreactor was disclosed many years later in WO2019/110710 with the drawback to be operated at very deep temperature of −50° C., to yield 98% of the 1,2-addition product mixture. This mixture then was treated with tetrabutylammonium hydroxide in aqueous solution to yield 92% FCTFE but with the disadvantage of much environmental unfriendly salt and waste water formation.

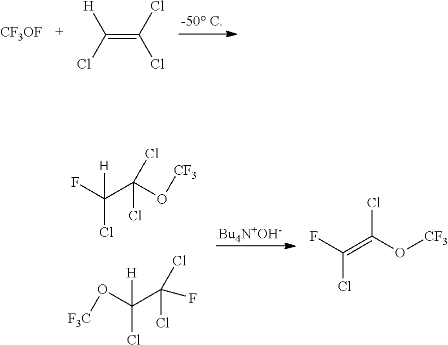

For PFMVE, the FCTFE was subjected in an additional step to an addition of F₂ and a dehydrohalogenation reaction, latter disclosed also already by Solvay Specialty Polymers in in WO2012/104365.

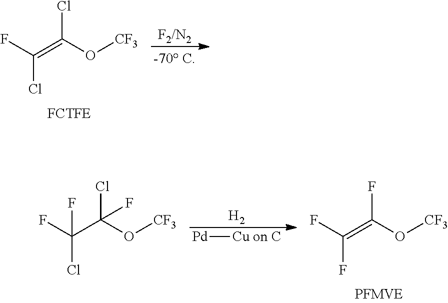

Good selectivities were reported for all steps but two steps are with deep temperature reactions, one step with waste water and salt formation and one step in gas phase, all of these steps have very high energy consumption and might have some economic limitations in industrial scale.

As shown herein before the prior art processes are not yet optimal and have several disadvantages. Such disadvantages of the prior art processes, for example, in particular encompass salt formation and high energy consumption. The high energy consumption in the prior art processes, e.g., is due to reaction step sequences requiring cooling in one step (liquid phase reaction step) and heating in another step (gas phase reaction step).

Accordingly, there is a high demand of enabling large-scale and/or industrial production of perfluoro(methyl vinyl ether) (PFMVE) and/or of 2-fluoro-1,2-dichloro-trifluoro-methoxyethylene (FCTFE) which is a suitable intermediate in the manufacture of perfluoro(methyl vinyl ether) (PFMVE), wherein the manufacture of PFMVE and/or FCTFE avoids the disadvantages of the prior art processes, and in particular does not encompass salt formation and has particularly less energy consumption than said prior art processes.

Thus, it is an object of the present invention to provide an efficient and simplified new industrial process for manufacturing of perfluoro(methyl vinyl ether) (PFMVE), and/or of 2-fluoro-1,2-dichloro-trifluoro-methoxyethylene (FCTFE).

It is a further object of the present invention to also provide an efficient and simplified new industrial process for manufacturing of perfluoro(methyl vinyl ether) (PFMVE) out of 2-fluoro-1,2-dichloro-trifluoro-methoxyethylene (FCTFE).

It is preferably another object of the present invention to provide an efficient and simplified new industrial process for manufacturing of perfluoro(methyl vinyl ether) (PFMVE), and/or of 2-fluoro-1,2-dichloro-trifluoro-methoxyethylene (FCTFE), and preferably enabling large-scale and/or industrial production of PFMVE and/or FCTFE by means of special equipment and special reactor design.

### SUMMARY OF THE INVENTION

The objects of the invention are solved as defined in the claims, and described herein after in detail.

Please also refer to drawing:

In FIG. 1, the first microreactor is a SiC-microreactor for addition (A) reaction, and the second microreactor is a Ni-microreactor for elimination (B) reaction. See reaction Scheme 3 below and Example 2. The CF₃OF-gas feed and the trifluoroethylene-gas feed are introduced in a first step for performing an addition (A) reaction as described below, and to obtain an addition product (A-P). In a second step the addition product (A-P) is subjected to an elimination (B) reaction to yield the product PFMVE which is collected in a cooling trap. The HF formed in the elimination (B) reaction (second step) leaves as purge gas over a cyclone as described herein.

In FIG. 2, see reaction Scheme 1 below and Example 3. The CF₃OF-gas feed and the trifluoroethylene-gas feed are introduced in a first step for performing an addition (A) reaction as described below, and to obtain an addition product (A-P). In a second step the addition product (A-P) is subjected to an elimination (B) reaction to yield the product FCTFE which is collected in a cooling trap. The HCl formed in the elimination (B) reaction (second step) leaves as purge gas over a cyclone as described herein.

In FIG. 3, see reaction Scheme 2 below and Example 6 to 8, and in particular Example 6. The FCTFE and the Lewis acid catalyst are withdrawn from each of their reservoir and are fed together into a mixer, and then the mixture thereof is passed on into a microreactor for fluorination (C) reaction, as described below, and to obtain the PFMVE product, which is collected in a cooling trap. The HCl formed in the fluorination (C) reaction leaves as purge gas over a cyclone as described herein. In these fluorination reactions liquid HF is dosed (fluorinating agent), especially anhydrous HF (hydrogen fluoride) or water-free HF (hydrogen fluoride), respectively.

In FIG. 4, two step batch process in a counter-current system. See reaction Scheme 1 below and Example 9. The reservoir is containing the liquid raw material trichloroethylene for the first step. The CF₃OF-gas feed is introduced in a first step for performing an addition (A) reaction as described below, and to obtain an addition product (A-P). In a second step (not shown) the addition product (A-P) is subjected to an elimination (B) reaction to yield the product FCTFE which is collected in a cooling trap. The HCl formed in the elimination (B) reaction (second step) leaves as purge gas during second step reaction together with inert gas used for purging the reactor system as described herein.

The invention relates to a new industrial process for manufacturing of perfluoro(methylvinylether) (PFMVE), and/or of 2-fluoro-1,2-dichloro-trifluoro-methoxyethylene (FCTFE), also known as 1,2-dichloro-1-fluoro-2-(trifluoromethoxy)-ethene (CAS number: 94720-91-9), which is a suitable intermediate in the manufacture of perfluoro(methylvinylether) (PFMVE), involving reactions in liquid phase and, for example, performing reactions in a microreactor, as each described here under and in the claims. The invention also relates to a new industrial process for manufacturing of perfluoro(methyl vinyl ether) (PFMVE) by fluorination, i.e., perfluorination, of 2-fluoro-1,2-dichloro-trifluoromethoxy-ethylene (FCTFE) with HF (hydrogen fluoride) in the presence of a Lewis acid catalyst, again performing the reaction in liquid phase, and preferably in a microreactor, as each described here under and in the claims.

For example, the present invention circumvents the mentioned disadvantages of the prior art processes, for example, the disadvantages of salt formation and high energy consumption. The high energy consumption in the prior art processes, e.g., is due to reaction step sequences requiring cooling in one step (liquid phase reaction step) and heating in another step (gas phase reaction step).

In contrast to the prior art processes, the reaction step sequences according to the present invention, by exemplification but not intended to be limited to this example, avoid such undesired salt formation and undesired high energy consumption by using, for example (representatively), CF₃OF (e.g., pre-prepared (in situ) by mixing COF₂ and F₂ in stoichiometric amounts) as a staring material, and reacting of CF₃OF, for example (representatively), with trichloroethylene (Cl₂C═CHCl).

Advantageously, according to the present invention, the addition (A) and elimination (B) reaction sequence can be performed without any conventional catalyst as used in the prior art, in this (representative) example of reacting CF₃OF with trichloroethylene (Cl₂C═CHCl; “Tri”) to yield an addition product (A-P) thereof, which in this (representative) example then is subjected to a dehydrohalogenation.

Dehydrohalogenation is elimination reaction that eliminates (removes) a hydrogen halide (H-Hal) from a substrate. Hydrogen halides (H-Hal) are known to be diatomic inorganic compounds with the formula H-Hal where “Hal” is one of the halogens, for example, fluorine or chlorine in the context or the present invention. Hydrogen halides, for example, such as in the present invention HF (hydrogen fluoride) or HCl (hydrogen chloride) are gases (under ambient conditions). In this (representative) example of the present invention substrate of the said dehydrohalogenation is the addition product (A-P) of CF₃OF and trichloroethylene (Cl₂C═CHCl), and the hydrogen halide which in the elimination (B) reaction of the present invention is eliminated (removed) from the said addition product (A-P) is HCl (hydrogen chloride), in this (representative) example then to yield the compound of formula (II), i.e., 2-fluoro-1,2-dichloro-trifluoro-methoxyethylene (FCTFE), which, as said before, is a suitable intermediate in the manufacture of perfluoro(methylvinylether) (PFMVE), i.e., the compound of formula (I).

Preferably, according to the present invention, the elimination (B) reaction can be performed in a Ni-reactor or in a reactor with a surface with high Ni-content (e.g., a Hastelloy steel) in liquid phase at 100° C. to easily yield the HCl-elimination product 1-chloro-1-fluoro-2-chloro-2-trifluoromethoxyethylene (FCTFE); see formula (I). This elimination (B) reaction proceeds most probably over the not evidenced and not isolated intermediates trifluoromethoxy trichlorofluoroethanes as shown in the reaction Scheme 1.

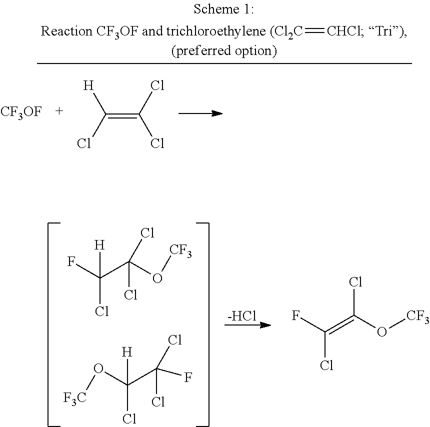

The HCl-elimination product 1-chloro-1-fluoro-2-chloro-2-trifluoromethoxyethylene (FCTFE) obtained according to reaction Scheme 1 is further reacted (e.g., subjected to a fluorination reaction) with HF and under Lewis acid catalysis. It is supposed that this fluorination (C) reaction occurs via the (neither evidenced nor isolated intermediates) 1,2-dichloro-2,2-difluoroethyl-trifluoromethyl-ether and 1,2-dichloro-1,2-difluoroethyl-trifluoromethyl-ether, as shown in the reaction Scheme 2, to finally yield perfluoro(methylvinylether) (PFMVE), i.e., the compound of formula (I).

The fluorination reaction with HF (hydrogen fluoride) according to the present invention is preferably performed in that liquid HF (the fluorinating agent), especially anhydrous HF (hydrogen fluoride) or water-free HF (hydrogen fluoride), respectively, is dosed into the reaction under Lewis acid catalysis.

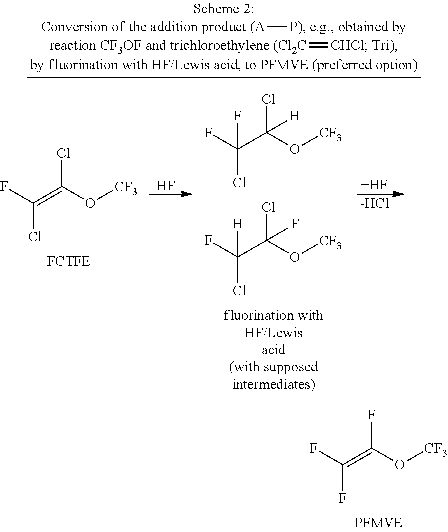

In comparison to Solvay's processes as identified above in the Background Section, this exemplified (preferred) reaction route of the invention according to Schemes 1 and/or 2 to yield F CTFE compound of formula (II) and/or to yield PFMVE compound of formula (I), as said before, is avoiding very deep temperatures, is two steps shorter than the prior art processes, avoids undesired salt formation and avoids waste water formation. In addition, as another big advantage, the exemplified (preferred) reaction route of the invention according to Schemes 1 and/or 2 uses much cheaper HF as a fluorination agent instead of expensive elemental fluorine (F₂), e.g., F₂-gas generated by electrolysis, for further fluorination of FCTFE compound of formula (II) to finally yield PFMVE compound of formula (I).

In contrast to the prior art processes, the reaction step sequences according to the present invention, by further exemplification as shown in reaction Scheme 3, but not intended to be limited to this further example of reaction Scheme 3 (an alternative preferred option), again is avoiding undesired salt formation and undesired high energy consumption by using, for example (representatively), CF₃OF (e.g., pre-prepared (in situ) by mixing COF₂ and F₂ in stoichiometric amounts) as a staring material, and reacting of CF₃OF, for example (representatively), with trifluoroethylene (F₂C═CHF), then directly yielding the perfluoro(methyl vinyl ether) (PFMVE), i.e., the compound of formula (I).

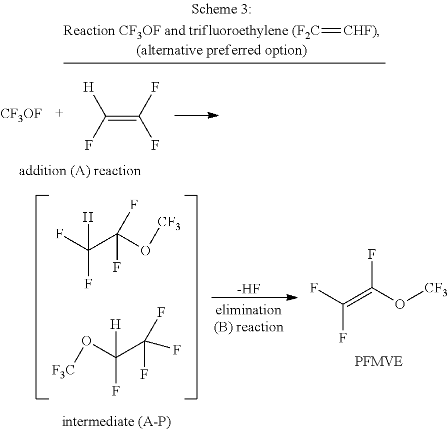

The reaction step sequences according to the present invention, by further exemplification as shown in reaction Scheme 4, but not intended to be limited to this further example of reaction Scheme 4 (a further alternative, but less preferred option), as mentioned here before, also shows advantages over the said processes of the prior art.

The alternative option, as shown in reaction Scheme 4 (addition (A) and elimination (B) reaction) and in reaction Scheme 5 (fluorination (C) reaction), is using CCl₃OCl alternatively to CF₃OF in the first addition (A) reaction step of the process according to the invention. This alternative option is comprised by the present invention, but somehow is less preferred, as either presence of higher amounts of chlorodifluoromethoxy vinyl fluoride must be accepted due to uncomplete fluorination of CCl₃— group to CF₂Cl group only. However, of course undesired CF₂Cl compound can be recovered, but this will need additional efforts of recycling and re-feeding into the reactor system, as compared to the preferred use of the before described CF₃OF. The CCl₃OCl can be prepared, for example, (in situ) in a microreactor by simply mixing COCl₂ with Cl₂, but complete conversion to CCl₃OCl requires almost a triple residence time as compared to preparing the preferred CF₃OF, for example, (in situ) by simply mixing COF₂ and F₂.

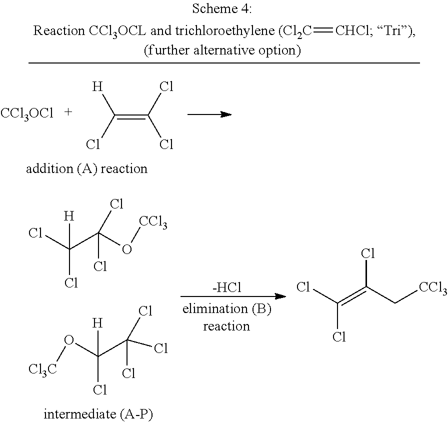

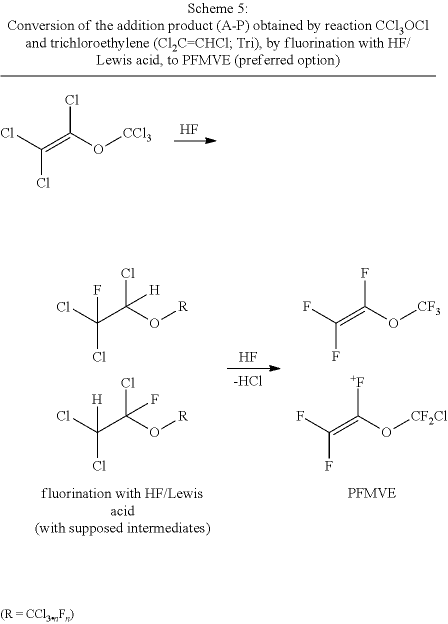

Firstly, having exemplified the invention here before, the process of the present invention, more generally, the present invention, is directed to a process for the manufacture of PFMVE (perfluoro(methyl vinyl ether)) having the formula (I),

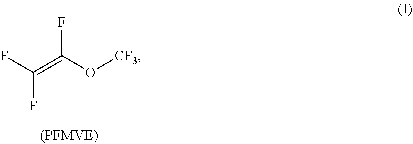

characterized in that a trihalomethyl hypohalogenite of formula (III) and a trihaloethylene of formula (IV) are reacted with each other,

CX₃—O—X (III),

wherein, in formula (III), X represents F (fluorine atom) or Cl (chlorine atom),

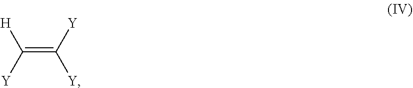

wherein, in formula (IV), Y represents F (fluorine atom) or Cl (chlorine atom);

and wherein the process comprises the steps of performing:

(A) in a first step in a first reactor, with the proviso that if the trihaloethylene of formula (IV) is a gaseous starting material then the first reactor is not a loop reactor, preferably wherein the first reactor is a microreactor, an addition reaction, wherein the trihalomethyl hypohalogenite of formula (III) is added to the trihaloethylene of formula (IV) and the addition reaction is performed at a temperature in the range of about 0° C. to about 35° C. to form an addition product (A-P); and subsequently,

with or without isolating the (liquid) addition product (A-P); preferably without isolating the (liquid) addition product (A-P),

(B) in a second step in said first reactor if the first reactor is a loop reactor, or in a second a reactor, which is a microreactor, in liquid phase an elimination reaction, wherein HY (hydrogen halogenide) is eliminated from the addition product (A-P) and the elimination reaction is performed at a temperature in the range of about 80° C. to about 120° C. to yield a trihalomethoxytrihaloethylene compound of formula (V),

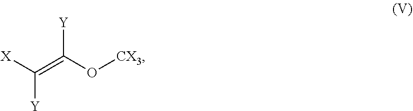

wherein in formula (V), X represents F (fluorine atom) or Cl (chlorine atom), Y represents F (fluorine atom) or Cl (chlorine atom);

and with the provisos (i) and (ii) that

(i) if X and Y are the same in each of the compounds of formulae (III) to (V), and each of X and Y represents F (fluorine atom), directly the compound of formula (I), PFMVE (perfluoro(methyl vinyl ether)), is obtained; and

(ii) if X and Y are different from each other in that either X represents F (fluorine atom) and Y represents Cl (chlorine atom), or X represents Cl (chlorine atom) and Y represents F (fluorine atom),

(C) then in a third reactor, preferably wherein the third reactor is microreactor, the trihalomethoxy trihaloethylene compound of formula (V) is subjected to a fluorination reaction in liquid phase, wherein the trihalomethoxy trihaloethylene compound of formula (V) is fluorinated with HF (hydrogen fluoride) in the presence of at least one Lewis acid catalyst, and at a temperature in the range of about 50° C. to about 100° C., in order to replace the Cl (chlorine atom) substituents contained in the compound of formula (V) by F (fluorine atom), by addition of HF and elimination of HCl (hydrogen chloride), and thereby to obtain the compound of formula (I), PFMVE (perfluoro(methyl vinyl ether)).

Secondly, having exemplified the invention here before, the process of the present invention, more generally, the present invention, is directed to a process for the manufacture of FCTFE (2-fluoro-1,2-dichloro-trifluoro-methoxyethylene) having the formula (II),

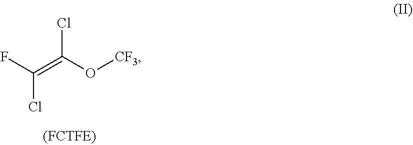

characterized in that a trifluoromethyl hypofluorite of formula (IIIa) and a trichloroethylene of formula (IVb) are reacted with each other,

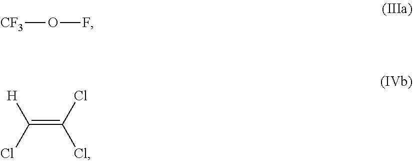 [SMILES: FOC(F)(F)F ; ClC=C(Cl)Cl]

and wherein the process comprises the steps of performing:

(A) in a first step in a first reactor, preferably in a loop reactor or in a micro reactor, more preferably in a microreactor, an addition reaction, wherein the trifluoromethyl hypofluorite of formula (IIIa) is added to the trichloroethylene of formula (IVb) and the addition reaction is performed at a temperature in the range of about 0° C. to about 35° C. to form an addition product (A-Pab); and subsequently,

with or without isolating the (liquid) addition product (A-P); preferably without isolating the (liquid) addition product (A-P),

(B) in a second step in said first reactor if the first reactor is a loop reactor, or in a second a reactor, which is a microreactor, in liquid phase an elimination reaction, wherein HCl (hydrogen chloride) is eliminated from the addition product (A-Pab) and the elimination reaction is performed at a temperature in the range of about 80° C. to about 120° C. to obtain the compound of formula (II), FCTFE (2-fluoro-1,2-dichloro-trifluoro-methoxyethylene).

The reaction steps (A) (addition reaction), (B) (elimination reaction; H-Hal elimination; H=hydrogen, Hal=halogen atom, i.e. fluorine or chlorine; hydrogen halogenide elimination) and/or (C) (fluorination step; with HF as fluorination agent in the presence of a Lewis acid catalyst) in the processes according to the present invention, described herein and in the claims, may be performed in various reactor designs. Example reactor designs include, a loop reactor system, a counter-current (loop) system (“inverse gas scrubber system”), a microreactor system (may include one or more), and coil reactor design. Particular reactor designs are shown in the FIG. 4 (gas scrubber system, counter-current [loop] system), FIGS. 1 to 3 (microreactor systems). Further, the fluorination step in the process of the invention may be performed in a batch or in a continuous manner, respectively. Further, any of the addition step (A), elimination step (B) and the fluorination step (C) in the process of the invention may be performed in a batch or in a continuous manner, respectively.

A preferred reactor used in any one of the steps (A) to (C), e.g., in one or more or in all steps of (A) to (C), of the present invention independently is a microreactor system. Preferably, in case of the step (B) (elimination reaction; H-Hal elimination), the reactor is a microreactor system (may include one or more).

With the exception when all starting materials of any of the reaction steps (A) to (C) are gaseous, any one of the steps (A) and (C) of the present invention independently may also be performed in a loop reactor system, a counter-current (loop) system (“inverse gas scrubber system”).

For example, if CF₃OF and trifluoroethylene, i.e., two gases are used as the starting materials, the addition reaction (A) at least initially will occur in the gas phase (gas phase reaction) until at least some (liquid) addition product (A-P) is formed. In such a case when gases are used as the starting materials in the reaction steps (A) to (C), the reactor is not a loop reactor system, a counter-current (loop) system (“inverse gas scrubber system”), but the reactor is microreactor system (may include one or more). See FIG. 1 (microreactor system).

For example, if CF₃OF and trichloroethylene, i.e., a gas (CF₃OF) and a liquid (trichloroethylene), are used as the starting materials, the addition reaction (A) will occur in the liquid phase, and the (liquid) addition product (A-P) is formed. In such a case when at least one liquid starting materials is used in the reaction steps (A) to (C), the reactor may also be a loop reactor system, a counter-current (loop) system (“inverse gas scrubber system”), but preferably also in this case the reactor is microreactor system (may include one or more). See FIG. 4 (gas scrubber system, counter-current [loop] system).

In case of a continuous manner process, i.e., when the continuous process according to the invention is performed in any one of the steps (A) to (C), e.g., in one or more or in all steps of (A) to (C), of the present invention independently reactor system is a microreactor system (may include one or more), as described herein and in the claims, and used in continuous operating manner.

In case of a batch manner process, and the starting materials are not gaseous, the batch process according to the invention can also be performed in a counter-current system, preferably as described herein and in the claims, in batch operating manner.

The invention also relates to process steps (A), (B), and/or (C), independently, as described herein and in the claims, optionally either operated in a batch manner or operated in a continuous manner, for the manufacture of the compound perfluoro(methyl vinylether) (PFMVE), and/or of the compound 2-fluoro-1,2-dichloro-trifluoro-methoxyethylene (FCTFE), i.e., the precursor or intermediate compound of perfluoro(methyl vinyl ether) (PFMVE), respectively, as each defined herein and in the claims, wherein the reaction is carried out in at least one step as a continuous processes, wherein the continuous process is performed in at least one continuous flow reactor with upper lateral dimensions of about ≤5 mm, or of about ≤4 mm,

preferably in at least one microreactor;

more preferably wherein of the said steps at least (b2) the step of a fluorination reaction is a continuous process in at least one microreactor under one or more of the following conditions:
flow rate: of from about 10 ml/h up to about 400 l/h;
temperature: ranging of from about −20° C. or of from about −10° C. or of from about 0° C. or of from about 10° C., or of from about 20° C. or of from about 30° C., respectively, each ranging to up to about 150° C.;
pressure: of from about 1 bar (1 atm abs.) up to about 50 bar; preferably of from about 1 bar (1 atm abs.) up to about 20 bar, more preferably at about 1 bar (1 atm abs.) up to about 5 bar; most preferably at about 1 bar (1 atm abs.) up to about 4 bar; in an example the pressure is about 3 bar;
residence time: of from about 1 second, preferably from about 1 minute, up to about 60 minutes.

The invention also relates to a process, as described herein, optionally either operated in a batch manner or operated in a continuous manner, for the manufacture of PFMVE (perfluoro(methyl vinyl ether)) having the formula (I), or process for the manufacture of FCTFE (2-fluoro-1,2-dichloro-trifluoro-methoxyethylene) having the formula (II), characterized in that in step (A) the in the first reactor the addition reaction is performed in an SiC-reactor.

The invention also relates to a process, as described herein, optionally either operated in a batch manner or operated in a continuous manner, for the manufacture of PFMVE (perfluoro(methyl vinyl ether)) having the formula (I), or a process for the manufacture of FCTFE (2-fluoro-1,2-dichloro-trifluoro-methoxyethylene) having the formula (II), characterized in that in step (B) in the second reactor the elimination reaction is performed in a nickel-reactor (Ni-reactor) or in a reactor with an inner surface with high nickel-content (Ni-content).

The boiling point of the compound perfluoro(methyl vinyl ether) (PFMVE) is −22° C. (at normal or ambient pressure), and thus, at room temperature the compound perfluoro-(methyl vinyl ether)(PFMVE) is gaseous. Accordingly, in an embodiment of the process of the invention the compound perfluoro(methyl vinyl ether) (PFMVE) is isolated in that there is a cooler used after the reaction, e.g., after the elimination step (B) reactor or after the fluorination step (C) reactor, to cool down the reaction mixture to 0° C. (cooler not shown in the Figures), and further in that most of the HF formed, e.g., in the elimination step (B) or most of the HCl formed in the fluorination step (C), is purged over a cyclone into a scrubber, and the compound perfluoro(methylvinylether) (PFMVE) is collected in a cooling trap kept at a temperature of below the boiling point of PFMVE, for example, at or below the boiling point of PFMVE which is about −22° C. (the cooling trap is also not shown in the Figures). For example, the cooling trap is kept at a temperature of about −30° C.

The boiling point of the compound 2-fluoro-1,2-dichloro-trifluoro-methoxyethylene (FCTFE), also known as 1,2-dichloro-1-fluoro-2-(trifluoromethoxy)-ethene (CAS number: 94720-91-9), i.e., of the precursor or intermediate compound of perfluoro(methyl vinyl ether) (PFMVE), is about 90.0° C.±40.0° C. (at normal or ambient pressure; predicted, source SciFinder®), and thus, at room temperature the compound 2-fluoro-1,2-dichloro-trifluoro-methoxyethylene (FCTFE) is liquid. Accordingly, in an embodiment of the process of the invention the compound 2-fluoro-1,2-dichloro-trifluoro-methoxyethylene (FCTFE) is isolated in that there is a cooler used after the reaction, e.g., after the elimination step (B) reactor, to cool down the reaction mixture to 0° C. (cooler not shown in the Figures), and further in that most of the HCl formed in the fluorination step (C), is purged over a cyclone into a scrubber, and the compound perfluoro(methyl vinyl ether) (PFMVE) is collected in a (cooling) trap kept at a temperature of below the boiling point of FCTFE, for example, sufficiently below the boiling point of FCTFE, for example, at about ambient temperature or about room temperature, respectively, e.g., at a temperature of about 25° C.; but lower temperatures than about ambient or room temperature are of course possible, too, e.g., a temperature of about 0° C., or if desired even below 0° C. (the [cooling] trap is also not shown in the Fig.s). The compound FCTFE (2-dichloro-1-fluoro-2-(trifluoromethoxy)-ethene) is also known, for example, under the following alternative names: 1,2-dichloro-1-fluoro-2-(trifluoromethoxy)ethene; and 1,2-dichloro-1-fluoro-2-trifluoromethoxyethene.

Characteristics of particular further compounds preferably used in the context of the present invention shall be exemplified.

Hypofluorites are formally derivatives of OF⁻, which is the conjugate base of hypofluorous acid. One example is trifluoromethyl hypofluorite (CF₃OF).

CF₃OF, trifluoromethyl hypofluorous acid ester, (CAS number: 373-91-1; the boiling point is about −94.2° C. at normal or ambient pressure; experimental, source SciFinder®), and thus, at room temperature the (starting material) compound CF₃OF is gaseous. The manufacture of CF₃OF, trifluoromethyl hypofluorous acid ester, is known in the technical art, and CF₃OF, trifluoromethyl hypofluorous acid ester, can be made (in situ) by simply mixing stoichiometric amounts of COF₂ (carbonyl difluoride; CAS number: 353-50-4; gaseous, boiling point −94.6° C., at normal or ambient pressure) and F₂ (elemental fluorine; gaseous). The compound CF₃OF (trifluoromethyl hypofluorous acid ester) is also known, for example, under the following alternative names: trifluoromethyl hypofluorite; trifluoro(fluorooxy)methane (trifluorofluoroxymethane); fluorooxytrifluoromethane (fluoroxytrifluoromethane); fluorooxyperfluoromethane.

CF₃OF, trifluoromethyl hypofluorous acid ester, is a derivative of hypofluorous acid (HOF), chemical formula HOF, is the only known oxoacid of fluorine and the only known oxoacid which the main atom gains electrons from oxygen to create a negative oxidation state. The oxidation state of the oxygen in hypofluorites is 0.

A related compound to the before said hypofluorous acid (HOF) is hypochlorous acid (HOCl) that is more technologically important but has not been obtained in pure form.

CCl₃OCl, trichloromethyl hypochlorous acid ester, (CAS number: 51770-65-1); the boiling point is about 142.9° C.±30.0° C. at normal or ambient pressure; predicted, source SciFinder®), and thus, at room temperature the (starting material) compound CCl₃OCl is liquid. The compound CCl₃OCl (trichloromethyl hypochlorous acid ester) is also known, for example, under the following alternative name: trichloromethyl hypochlorite. The manufacture of CCl₃OCl, trichloromethyl hypochlorous acid ester, is known in the technical art. Analogously to trifluoromethyl hypofluorite (CF₃OF), the compound CCl₃OCl, trichloromethyl hypochlorous acid ester, can be made (in situ) by simply mixing stoichiometric amounts of COCl₂ (carbonyl dichloride, also known as phosgene; CAS number: 75-44-5; gaseous, boiling point 7.4° C., at normal or ambient pressure) and C12 (elemental chlorine; gaseous).

Trifluoroethylene (CAS number: 359-11-5); the boiling point is about −53.0° C. (starting at about −51.0° C.), at normal or ambient pressure, and thus, at room temperature the (starting material) compound trichloroethylene is gaseous. The compound trifluoroethylene is also known, for example, under the following alternative name: trifluoroethene; ethylene trifluoride. The manufacture of trifluoroethylene is well known in the technical art.

Trichloroethylene (CAS number: 79-01-6); the boiling point is about 87.0° C. at normal or ambient pressure, and thus, at room temperature the (starting material) compound trichloroethylene is liquid. The compound trichloroethylene is also known, for example, under the following alternative names: ethylene trichloride; trichlorethene; TCE; Tri. The manufacture of trichloroethylene is well known in the technical art.

### BRIEF DESCRIPTION OF THE DRAWINGS

FIG. 1 shows the manufacture of PFMVE by reaction of CF₃OF with trifluoroethylene in a sequence of two microreactors.

FIG. 2 shows the manufacture of FCTFE by reaction of CF₃OF with trichloroethylene in a sequence of two microreactors.

FIG. 3 shows the manufacture of PFMVE out of FCTFE by fluorination with HF in the presence of a Lewis acid catalyst in a microreactor.

FIG. 4 shows the manufacture of FCTFE out of trichloroethylene and CF₃OF using a gas scrubber system.

### DETAILED DESCRIPTION OF THE INVENTION

As briefly described in the Summary of the Invention, and defined in the claims and further detailed by the following description and examples herein, the invention relates to a new industrial process for manufacturing of perfluoro(methyl vinyl ether) (PFMVE), and/or of 2-fluoro-1,2-dichloro-trifluoro-methoxyethylene (FCTFE), also known as 1,2-dichloro-1-fluoro-2-(trifluoromethoxy)-ethene (CAS number: 94720-91-9), which is a suitable intermediate in the manufacture of perfluoro(methyl vinyl ether) (PFMVE), involving reactions in liquid phase and performing reactions in a microreactor, as each described here under and in the claims.

The invention particularly also relates to a new industrial process for manufacturing of perfluoro(methyl vinyl ether) (PFMVE) by fluorination, i.e., perfluorination, of 2-fluoro-1,2-dichloro-trifluoromethoxy-ethylene (FCTFE) with HF (hydrogen fluoride) in the presence of a Lewis acid catalyst, again performing the reaction in liquid phase, and preferably in a microreactor, as each described here under and in the claims.

In a first aspect, the invention pertains to a process for the manufacture of PFMVE (perfluoro(methyl vinyl ether)) having the formula (I),

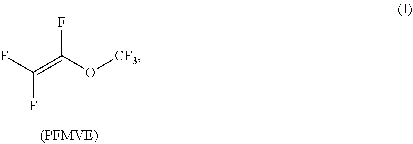

characterized in that a trihalomethyl hypohalogenite of formula (III) and a trihaloethylene of formula (IV) are reacted with each other,

CX3-O—X (III),

wherein, in formula (III), X represents F (fluorine atom) or Cl (chlorine atom),

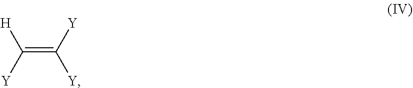

wherein, in formula (IV), Y represents F (fluorine atom) or Cl (chlorine atom);

and wherein the process comprises the steps of performing:

(A) in a first step in a first reactor, with the proviso that if the trihaloethylene of formula (IV) is a gaseous starting material then the first reactor is not a loop reactor, preferably wherein the first reactor is a microreactor, an addition reaction, wherein the trihalomethyl hypohalogenite of formula (III) is added to the trihaloethylene of formula (IV) and the addition reaction is performed at a temperature in the range of about 0° C. to about 35° C. to form an addition product (A-P); and subsequently,

with or without isolating the (liquid) addition product (A-P); preferably without isolating the (liquid) addition product (A-P),

(B) in a second step in said first reactor if the first reactor is a loop reactor, or in a second a reactor, which is a microreactor, in liquid phase an elimination reaction, wherein HY (hydrogen halogenide) is eliminated from the addition product (A-P) and the elimination reaction is performed at a temperature in the range of about 80° C. to about 120° C. to yield a trihalomethoxy trihaloethylene compound of formula (V),

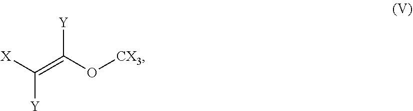

wherein in formula (V), X represents F (fluorine atom) or Cl (chlorine atom), Y represents F (fluorine atom) or Cl (chlorine atom);

and with the provisos (i) and (ii) that

(i) if X and Y are the same in each of the compounds of formulae (III) to (V), and each of X and Y represents F (fluorine atom), directly the compound of formula (I), PFMVE (perfluoro(methyl vinyl ether)), is obtained; and

(ii) if X and Y are different from each other in that either X represents F (fluorine atom) and Y represents Cl (chlorine atom), or X represents Cl (chlorine atom) and Y represents F (fluorine atom),

(C) then in a third reactor, preferably wherein the third reactor is microreactor, the trihalomethoxytrihaloethylene compound of formula (V) is subjected to a fluorination reaction in liquid phase, wherein the trihalomethoxytrihaloethylene compound of formula (V) is fluorinated with HF (hydrogen fluoride) in the presence of at least one Lewis acid catalyst, and at a temperature in the range of about 50° C. to about 100° C., in order to replace the Cl (chlorine atom) substituents contained in the compound of formula (V) by F (fluorine atom), by addition of HF and elimination of HCl (hydrogen chloride), and thereby to obtain the compound of formula (I), PFMVE (perfluoro(methyl vinyl ether)).

In another aspect, the invention pertains to a process as defined here before, for the manufacture of PFMVE (perfluoro(methyl vinyl ether)) having the formula (I), characterized in that X in the trihalomethyl hypohalogenite of formula (III) represents F (fluorine atom).

In this preferred aspect, the invention in particular pertains to a process as defined here before, for the manufacture of PFMVE (perfluoro(methyl vinyl ether)) having the formula (I),

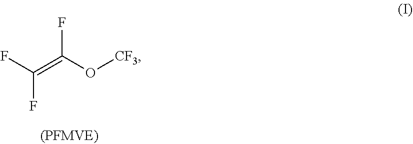

characterized in that a trifluoromethyl hypofluorite of formula (IIIa) and a trichloroethylene of formula (IV) are reacted with each other,

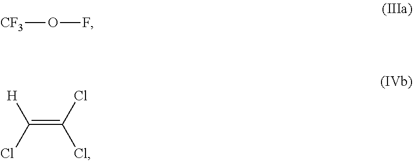 [SMILES: FOC(F)(F)F ; ClC=C(Cl)Cl]

and wherein the process comprises the steps of performing:

(A) in a first step in a first reactor, preferably in a loop reactor or in a micro reactor, more preferably in a microreactor, an addition reaction, wherein the trifluoromethyl hypofluorite of formula (IIIa) is added to the trichloroethylene of formula (IVb) and the addition reaction is performed at a temperature in the range of about 0° C. to about 35° C. to form an addition product (A-Pab); and subsequently, with or without isolating the (liquid) addition product (A-P); preferably without isolating the (liquid) addition product (A-P),

(B) in a second step in said first reactor if the first reactor is a loop reactor, or in a second a reactor, which is a microreactor, in liquid phase an elimination reaction, wherein HY (hydrogen halogenide) is eliminated from the addition product (A-Pab) and the elimination reaction is performed at a temperature in the range of about 80° C. to about 120° C. to yield a compound of formula (II) (2-fluoro-1,2-dichloro-trifluoro-methoxyethylene),

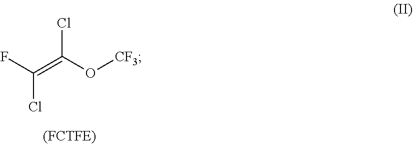

and

(C) then in a third reactor the compound of formula (II) (2-fluoro-1,2-dichloro-trifluoro-methoxyethylene) is subjected to a fluorination reaction in liquid phase, wherein the compound of formula (II) is fluorinated with HF (hydrogen fluoride) in the presence of at least one Lewis acid catalyst, and at a temperature in the range of about 50° C. to about 100° C., in order to replace the Cl (chlorine) atoms contained in the compound of formula (II) by F (fluorine) atoms, by addition of HF and elimination of HCl (hydrogen chloride), and thereby to obtain the compound of formula (I), PFMVE (perfluoro(methyl vinyl ether)).

In yet another aspect, the invention pertains to a process as defined here before, for the manufacture of PFMVE (perfluoro(methyl vinyl ether)) having the formula (I), characterized in that Y in the trihaloethylene of formula (IV) represents F (fluorine atom).

In still another aspect, the invention pertains to a process as defined here before, for the manufacture of PFMVE (perfluoro(methyl vinyl ether)) having the formula (I), characterized in that X in the trihalomethyl hypohalogenite of formula (III) and Y in the trihaloethylene of formula (IV) both represent F (fluorine atom).

In this alternatively preferred aspect, the invention in particular pertains to a process as defined here before, for the manufacture of PFMVE (perfluoro(methyl vinyl ether)) having the formula (I),

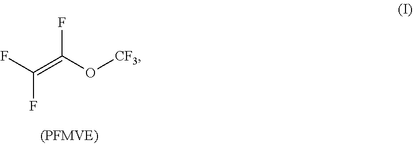

characterized in that a trifluoromethyl hypofluorite of formula (IIIa) and a trifluoroethylene of formula (IVa) are reacted with each other,

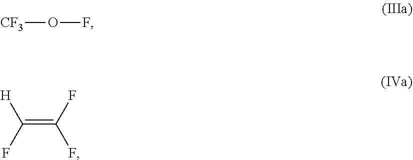 [SMILES: FOC(F)(F)F ; FC=C(F)F]

and wherein the process comprises the steps of performing:

(A) in a first step in a first reactor, with the proviso that (for the reason that the trifluoroethylene of formula (IVa) is a gaseous starting material) the first reactor is not a loop reactor, preferably wherein the first reactor is microreactor, an addition reaction, wherein the trifluoromethyl hypofluorite of formula (IIIa) is added to the trifluoroethylene of formula (IVa) and the addition reaction is performed at a temperature in the range of about 0° C. to about 35° C. to form an addition product (A-Paa); and subsequently,

with or without isolating the (liquid) addition product (A-P); preferably without isolating the (liquid) addition product (A-P),

(B) in a second step in a second reactor, preferably microreactor, in liquid phase an elimination reaction, wherein HF (hydrogen fluoride) is eliminated from the addition product (A-Paa) and the elimination reaction is performed at a temperature in the range of about 80° C. to about 120° C. to obtain the compound of formula (I), PFMVE (perfluoro(methyl vinyl ether)).

In a particular further aspect, the invention also pertains to a process as defined here before, for the manufacture of PFMVE (perfluoro(methyl vinyl ether)) having the formula (I),

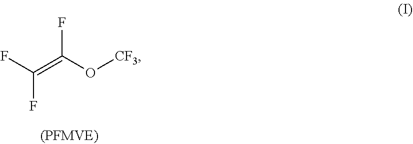

characterized in that the process comprises performing a step (C):

(C) wherein in a reactor, preferably wherein the reactor is microreactor, a compound of formula (II) (2-fluoro-1,2-dichloro-trifluoro-methoxyethylene),

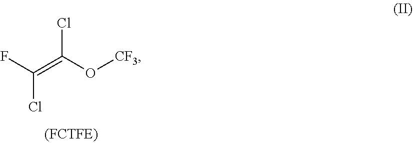

is subjected to a fluorination reaction in liquid phase, wherein the compound of formula (II) (2-fluoro-1,2-dichloro-trifluoro-methoxyethylene) is fluorinated with HF (hydrogen fluoride) in the presence of at least one Lewis acid catalyst, and at a temperature in the range of about 50° C. to about 100° C., in order to replace the Cl (chlorine) atoms contained in the compound of formula (II) by F (fluorine) atoms, by addition of HF and elimination of HCl (hydrogen chloride), and thereby to obtain the compound of formula (I), PFMVE (perfluoro(methyl vinyl ether)).

The present invention also pertains to a the manufacture of 2-fluoro-1,2-dichloro-trifluoro-methoxyethylene (FCTFE), also known as 1,2-dichloro-1-fluoro-2-(trifluoromethoxy)-ethene (CAS number: 94720-91-9), which is a suitable intermediate in the manufacture of perfluoro(methyl vinyl ether) (PFMVE). In this particular aspect, the present invention pertains to a process for the manufacture of FCTFE (2-fluoro-1,2-dichloro-trifluoro-methoxyethylene) having the formula (II),

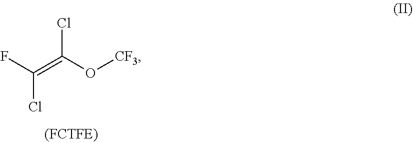

characterized in that a trifluoromethyl hypofluorite of formula (IIIa) and a trichloroethylene of formula (IVb) are reacted with each other,

CF₃—O—F (IIIa),

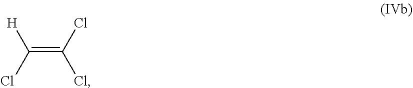

and wherein the process comprises the steps of performing:

(A) in a first step in a first reactor, preferably in a loop reactor or in a micro reactor, more preferably in a microreactor, an addition reaction, wherein the trifluoromethyl hypofluorite of formula (IIIa) is added to the trichloroethylene of formula (IVb) and the addition reaction is performed at a temperature in the range of about 0° C. to about 35° C. to form an addition product (A-Pab); and subsequently,

with or without isolating the (liquid) addition product (A-P); preferably without isolating the (liquid) addition product (A-P),

(B) in a second step in said first reactor if the first reactor is a loop reactor, or in a second a reactor, which is a microreactor, in liquid phase an elimination reaction, wherein HCl (hydrogen chloride) is eliminated from the addition product (A-Pab) and the elimination reaction is performed at a temperature in the range of about 80° C. to about 120° C. to obtain the compound of formula (II), FCTFE (2-fluoro-1,2-dichloro-trifluoro-methoxyethylene).

In a further aspect, the invention pertains also to any one of the above defined processes for the manufacture of PFMVE (perfluoro(methyl vinyl ether)) having the formula (I), or also to any one of the above defined processes for the manufacture of FCTFE (2-fluoro-1,2-dichloro-trifluoro-methoxyethylene) having the formula (II), characterized in that in step (A) in the first step in the first reactor the addition reaction is performed at a temperature in the range of about 15° C. to about 35° C. (or a temperature of about 25° C.±10° C.), preferably at a temperature in the range of about 20° C. to about 30° C. (or a temperature of about 25° C.±5° C.), more preferably at ambient (or room) temperature (or a temperature of about 20° C. to about 25° C.).

In yet a further aspect, the invention pertains also to any one of the above defined processes for the manufacture of PFMVE (perfluoro(methyl vinyl ether)) having the formula (I), or also to any one of the above defined processes for the manufacture of FCTFE (2-fluoro-1,2-dichloro-trifluoro-methoxyethylene) having the formula (II), characterized in that in step (B) in the second step in said first reactor if the first reactor is a loop reactor, or in a second reactor, which is a microreactor, the elimination reaction is performed at a temperature in the range of about 90° C. to about 110° C. (or a temperature of about 100° C.±10° C.), preferably at a temperature in the range of about 95° C. to about 105° C. (or a temperature of about 100° C.±5° C.), or at a temperature of about 100° C. (e.g., at a temperature of about 100° C.±4° C., or 100° C.±3° C., or 100° C.±2° C., or 100° C.±1° C.).

In a still a further aspect, the invention pertains also to any one of the above defined processes for the manufacture of PFMVE (perfluoro(methyl vinyl ether)) having the formula (I), characterized in that in step (C) the fluorination reaction is performed at a temperature in the range of about 50° C. to about 100° C., preferably at a temperature in the range of about 60° C. to about 100° C., more preferably at a temperature in the range of about 60° C. to about 90° C., even more preferably at a temperature in the range of about 70° C. to about 90° C. (or a temperature of about 80° C.±10° C.), still more preferably at a temperature in the range of about 70° C. to about 80° C. (or a temperature of about 100° C.±5° C.), or at a temperature of about 75° C. (e.g., at a temperature of about 75° C.±4° C., or 75° C.±3° C., or 75° C.±2° C., or 75° C.±1° C.).

In another aspect, the invention pertains also to any one of the above defined processes for the manufacture of PFMVE (perfluoro(methyl vinyl ether)) having the formula (I), or to any one of the above defined processes for the manufacture of FCTFE (2-fluoro-1,2-dichloro-trifluoro-methoxyethylene) having the formula (II), characterized in that prior to starting any of the process steps (A), (B), and (C) (if applicable), one or more of the reactors used, preferably each and any of the reactors used, are purged with an inert gas, preferably with He (helium) as the inert gas.

In yet another aspect, the invention pertains also to any one of the above defined processes for the manufacture of PFMVE (perfluoro(methyl vinyl ether)) having the formula (I), or to any one of the above defined processes for the manufacture of FCTFE (2-fluoro-1,2-dichloro-trifluoro-methoxyethylene) having the formula (II), characterized in that in step (A) the in the first reactor the addition reaction is performed in an SiC-reactor.

In still another aspect, the invention pertains also to any one of the above defined processes for the manufacture of PFMVE (perfluoro(methyl vinyl ether)) having the formula (I), or also to any one of the above defined processes for the manufacture of FCTFE (2-fluoro-1,2-dichloro-trifluoro-methoxyethylene) having the formula (II), characterized in that in step (B) in the second reactor the elimination reaction is performed in a nickel-reactor (Ni-reactor) or in a reactor with an inner surface with high nickel-content (Ni-content). Preferably, in the context of the present invention the term “high nickel-content” means a nickel (Ni) content of at least 50% in the metal alloy the nickel-reactor is made of Particularly preferred is a nickel-reactor made out of Hastelloy C4 nickel alloy. The Hastelloy C4 nickel alloy is known in the state of the art to be a nickel alloy comprising a combination of chromium with high molybdenum content. Such Hastelloy C4 nickel alloy shows exceptional resistance to a large number of chemical media such as contaminated, reducing mineral acids, chlorides and organic and inorganic media contaminated with chloride.

Hastelloy C4 nickel alloy is commercially available, for example, under the tradenames Nicrofer® 6616 hMo or Hastelloy C-4®, respectively. The density of Hastelloy C4 nickel alloy is 8.6 g/cm³, and the melting temperature range is 1335 to 1380° C.

Due to its special chemical composition of C4, the Hastelloy C4 nickel alloy has good structural stability and high resistance to sensitization.

The chemical composition of Hastelloy C4 (nickel alloy), for example, is in the following Table 1, wherein the nickel (Ni) content is at least 50% in the metal alloy, and the nickel (Ni) content is adding up the Hastelloy C4 nickel alloy compositions to a total of 100% metal alloy.

TABLE 1
Chemical composition of Hastelloy C4 (nickel alloy). and nickel
(Ni) as the remainder for adding up to 100% metal alloy.

| C % | Si ≤ % | Mn ≤ % | P ≤ % | S ≤ % | Cr % | Mo % | Co ≤ % | Fe % | Ti % |
|---|---|---|---|---|---|---|---|---|---|
| 0-.009 | 0-0.05 | 0-1.0 | 0-0.02 | 0-0.01 | 14.5-17.5 | 14.0-17.0 | 0-2.0 | 0-3 | 0-0.7 |

In a further aspect, the invention pertains also to any one of the above defined processes for the manufacture of PFMVE (perfluoro(methyl vinyl ether)) having the formula (I), characterized in that in step (C) the fluorination reaction is performed in a continuous manner, preferably in a continuous manner in a microreactor.

In yet a further aspect, the invention pertains also to any one of the above defined processes for the manufacture of PFMVE (perfluoro(methyl vinyl ether)) having the formula (I), characterized in that in step (C) the fluorination reaction is performed in the presence of a Lewis acid catalyst selected from the group consisting of SnCl₄ (tin tetrachloride), TiCl₄ (titanium tetrachloride), and SbF₅ (antimony pentafluoride).

In still a further aspect, the invention pertains also to any one of the above defined processes for the manufacture of PFMVE (perfluoro(methyl vinyl ether)) having the formula (I), characterized in that in step (C) the fluorination reaction is performed in the presence of the Lewis acid catalyst SbF₅ (antimony pentafluoride).

In a particular and preferred aspect, the invention pertains also to any one of the above defined processes for the manufacture of PFMVE (perfluoro(methyl vinyl ether)) having the formula (I), or also to any one of the above defined processes for the manufacture of FCTFE (2-fluoro-1,2-dichloro-trifluoro-methoxyethylene) having the formula (II), characterized in that that in step (A) the addition reaction is performed in a continuous manner, preferably in a continuous manner in a microreactor.

In another particular and preferred aspect, the invention pertains also to any one of the above defined processes for the manufacture of PFMVE (perfluoro(methyl vinyl ether)) having the formula (I), or also to any one of the above defined processes for the manufacture of FCTFE (2-fluoro-1,2-dichloro-trifluoro-methoxyethylene) having the formula (II), characterized in that in step (B) the elimination reaction is performed in a continuous manner, preferably in a continuous manner in a microreactor.

In yet another particular and preferred aspect, the invention pertains also to any one of the above defined processes for the manufacture of PFMVE (perfluoro(methyl vinyl ether)) having the formula (I), or also to any one of the above defined processes for the manufacture of FCTFE (2-fluoro-1,2-dichloro-trifluoro-methoxyethylene) having the formula (II), characterized in that, independently the reaction in at least one reaction step of (A), (B), and (C) (if applicable), is carried as a continuous processes, wherein the continuous process in the at least one reaction step of (A), (B), and (C) (if applicable), is performed in at least one continuous flow reactor with upper lateral dimensions of about ≤5 mm, or of about ≤4 mm, preferably wherein at least one of the continuous flow reactor is a microreactor.

In a more preferred aspect, the invention pertains also to any one of the above defined processes for the manufacture of PFMVE (perfluoro(methyl vinyl ether)) having the formula (I), or also to any one of the above defined processes for the manufacture of FCTFE (2-fluoro-1,2-dichloro-trifluoro-methoxyethylene) having the formula (II), characterized in that the reaction is carried out in at least one reaction step of (A), (B), and (C) (if applicable), as a continuous processes, wherein the continuous process is performed in at least one continuous flow reactor with upper lateral dimensions of about ≤5 mm, or of about ≤4 mm, preferably in at least one microreactor;

more preferably wherein of the said steps of (A), (B), and (C), at least the step (C) of a fluorination reaction is a continuous process in at least one microreactor under one or more of the following conditions:
flow rate: of from about 10 ml/h up to about 400 l/h;
temperature: ranging of from about −20° C. or of from about −10° C. or of from about 0° C. or of from about 10° C., or of from about 20° C. or of from about 30° C., respectively, each ranging to up to about 150° C.;
pressure: of from about 1 bar (1 atm abs.) up to about 50 bar; preferably of from about 1 bar (1 atm abs.) up to about 20 bar, more preferably at about 1 bar (1 atm abs.) up to about 5 bar; most preferably at about 1 bar (1 atm abs.) up to about 4 bar; in an example the pressure is about 3 bar;
residence time: of from about 1 second, preferably from about 1 minute, up to about 60 minutes.

In a further aspect, the invention pertains also to any one of the above defined processes for the manufacture of PFMVE (perfluoro(methyl vinyl ether)) having the formula (I), or also to any one of the above defined processes for the manufacture of FCTFE (2-fluoro-1,2-dichloro-trifluoro-methoxyethylene) having the formula (II), characterized in that, independently, the product yielding from step (A), the product resulting from step (B) and/or the product yielding from step (C) (if applicable) are subjected to distillation.

Batch Process:

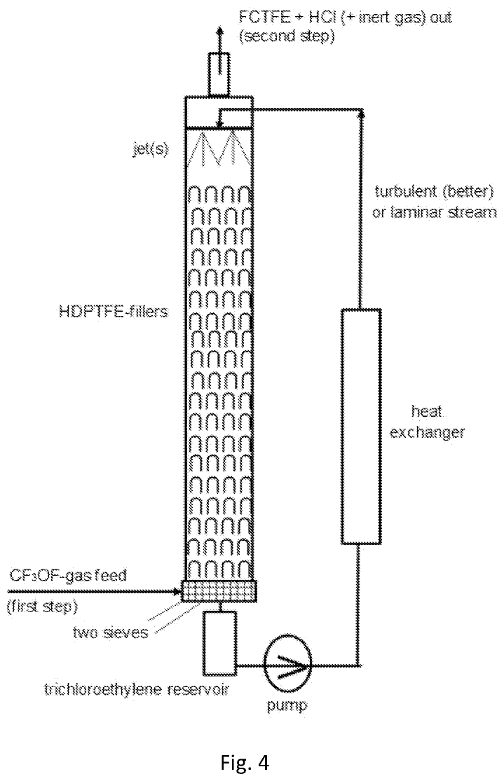

The invention also may pertain to a process for the manufacture of a fluorinated compound, comprising a particular process step which is performed batchwise, preferably wherein the batchwise process step is carried out in a column reactor. Although, in the following column reactor setting the process is described as a batch process, optionally the process can be performed in the said column reactor setting also as a continuous process. In case of a continuous process in the said column reactor setting, then, it goes without saying, the additional inlet(s) and outlet(s) are foreseen, for feeding the starting compound and withdrawing the product compound, respectively, and/or if desired any intermediate compound. Reference is made to FIG. 4 and Example 9.

If the invention pertains to a batchwise process, preferably wherein the batchwise process is carried out in a column reactor, the process for manufacturing of perfluoro(methylvinylether) (PFMVE), and/or of 2-fluoro-1,2-dichloro-trifluoro-methoxyethylene (FCTFE), also known as 1,2-dichloro-1-fluoro-2-(trifluoromethoxy)-ethene (CAS number: 94720-91-9), which is a suitable intermediate in the manufacture of perfluoro(methylvinylether) (PFMVE), most preferably the reaction is carried out in a (closed) column reactor (system), wherein the liquid medium comprising or consisting of a liquid starting compound, e.g., trichloroethylene or FCTFE, respectively, is circulated in a loop, while a gaseous starting compound, e.g., CF₃OF (trifluoromethyl hypofluorous acid ester) or a HF-fluorination gas, respectively, is fed into the column reactor and is passed through the liquid medium therein to react with the liquid starting compound; preferably wherein the loop in the column reactor is operated with a circulation velocity of from 1,500 l/h to 5,000 l/h, more preferably of from 3,500 l/h to 4,500 l/h.

If the invention pertains to such a batchwise process, the process for manufacturing of perfluoro(methyl vinyl ether) (PFMVE) and/or of 2-fluoro-1,2-dichloro-trifluoro-methoxyethylene (FCTFE) according to the invention can be carried out such that the mentioned liquid medium is circulated in the column reactor in a turbulent stream or in laminar stream, preferably in a turbulent stream.

In general, the gaseous starting compound, e.g., CF₃OF (trifluoromethyl hypofluorous acid ester) or a HF-fluorination gas, respectively, is fed into the loop in accordance with the required stoichiometry for the targeted product compound and/or if desired any intermediate compound, and adapted to the reaction rate.

For example, the said process for the manufacture of a compound PFMVE and/or FCTFE according to the invention, may be performed, e.g., batchwise, wherein the column reactor is equipped with at least one of the following: at least one cooler (system), at least one liquid reservoir for the liquid medium comprising or consisting of a liquid starting compound, a pump (for pumping/circulating the liquid medium), one or more (nozzle) jets, preferably placed at the top of the column reactor, for spraying the circulating medium into the column reactor, one or more feeding inlets for introducing a gaseous starting compound, e.g., CF₃OF (trifluoromethyl hypofluorous acid ester) or a HF-fluorination gas, respectively, optionally one or more sieves, preferably two sieves, preferably the one or more sieves placed at the bottom of the column reactor, and at least one gas outlet equipped with a pressure valve.

Accordingly, the process for manufacturing of perfluoro(methyl vinyl ether) (PFMVE) and/or of 2-fluoro-1,2-dichloro-trifluoro-methoxyethylene (FCTFE) compound according to the invention, can be performed in column reactor which is equipped with at least one of the following:

(i) at least one cooler (system), at least one liquid reservoir, with inlet and outlet for, and containing the liquid medium comprising or consisting of a starting compound; preferably trichloroethylene or FCTFE, respectively;

(ii) a pump for pumping and circulating the liquid medium in the column reactor;

(iii) one or more (nozzle) jets, preferably wherein the one or more (nozzle) jets are placed at the top of the column reactor, for spraying the circulating liquid medium into the column reactor;

(iv) one or more feeding inlets for introducing a gaseous starting compound, e.g., CF3OF (trifluoromethyl hypofluorous acid ester) or a HF-fluorination gas, respectively into the column reactor;

(v) optionally one or more sieves, preferably two sieves, preferably the one or more sieves placed at the bottom of the column reactor;

(vi) and at least one gas outlet equipped with a pressure valve, and at least one outlet for withdrawing the product compound, respectively, and/or if desired any intermediate compound.

In one embodiment, the process for manufacturing of perfluoro(methyl vinyl ether) (PFMVE) and/or of 2-fluoro-1,2-dichloro-trifluoro-methoxyethylene (FCTFE) compound according to the invention can be performed in a column reactor which is a packed bed tower reactor, preferably a packed bed tower reactor which is packed with fillers (the terms “filler” and “filling”, are meant synonymously in the context of the invention) resistant to the reactants and especially resistant to hydrogen fluoride (HF). Fillers resistant to the reactants and especially resistant to hydrogen fluoride (HF) suitable in the context of the present invention are in particular HF-resistant plastic fillers and/or HF-resistant metal fillers. For example, under certain circumstances the packed bed tower reactor may be packed with stainless steel (1.4571) fillers, but stainless steel (1.4571) fillers are less suitable than other fillers mentioned herein after, because of possible risk of (minor) traces of humidity in the reactor system. Preferably, for example, in the invention the packed bed tower reactor is packed with fillers resistant to the reactants and especially resistant to hydrogen fluoride (HF) such as, e.g., with Raschig fillers, E-TFE fillers, and/or HF-resistant metal fillers, e.g., Hastelloy metal fillers, and/or (preferably) HDPTFE-fillers, more preferably wherein the packed bed tower reactor is a gas scrubber system (tower) which is packed with any of the before mentioned HF-resistant Hastelloy metal fillers and/or HDPTFE-fillers, and preferably with HDPTFE-fillers.

In a further embodiment, the process for manufacturing of perfluoro(methyl vinyl ether) (PFMVE) and/or of 2-fluoro-1,2-dichloro-trifluoro-methoxyethylene (FCTFE) compound according to the invention, the reaction is carried out with a counter-current flow of the circulating liquid medium comprising or consisting of the liquid starting compound and of the gaseous starting compound or a HF-fluorination gas, respectively, that are fed into the column reactor.

The pressure valve functions to keep the pressure, as required in the reaction, and to release any effluent gas, e.g. inert carrier gas contained in the fluorination gas, if applicable together with any hydrogen halogenide gas released from the reaction.

The said process for manufacturing of perfluoro(methyl vinyl ether) (PFMVE) and/or of 2-fluoro-1,2-dichloro-trifluoro-methoxyethylene (FCTFE) compound according to the invention, may be performed, e.g., batchwise, such that in the said process the column reactor is a packed bed tower reactor as mentioned before, preferably a packed bed tower reactor which is packed with HDPTFE-fillers.

The packed tower according to FIG. 4 can have a diameter of 100 or 200 mm (depending on the circulating flow rate and scale) made out of Hastelloy C4 (nickel alloy)(known to the person skilled in the art), and has a length of 3 meters for the 100 mm and a length of 6 meters for the 200 mm diameter tower (latter if higher capacities are needed). The tower made out of Hastelloy is filled either with any of the fillings as mentioned before, or with the preferred HDPTFE-fillers, each of 10 mm diameter as commercially available. The size of fillings is quite flexible. The type of fillings is also quite flexible, within the boundaries of properties as stated herein above, i.e., the HDPTFE-fillers (or HDPTFE-fillings, respectively) were used in the trials disclosed hereunder in Example 9, and showed same performance, not causing much pressure reduction (pressure loss) while feeding any gaseous (starting) compound in counter-current manner.

Methods with Microreactor, Applicable Also to Variant with Coiled Reactor:

According to a preferred embodiment of the present invention, the compound perfluoro(methylvinylether) (PFMVE) and/or the compound 2-fluoro-1,2-dichloro-trifluoro-methoxyethylene (FCTFE), respectively, can also be prepared in a continuous manner. More preferably, the compound perfluoro(methyl vinyl ether) (PFMVE) and/or the compound 2-fluoro-1,2-dichloro-trifluoro-methoxyethylene (FCTFE), respectively, in microreactor reaction.

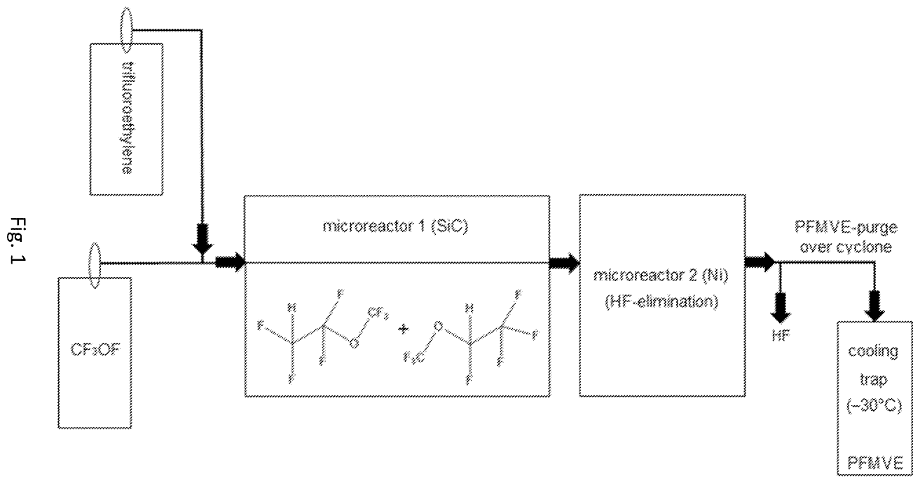

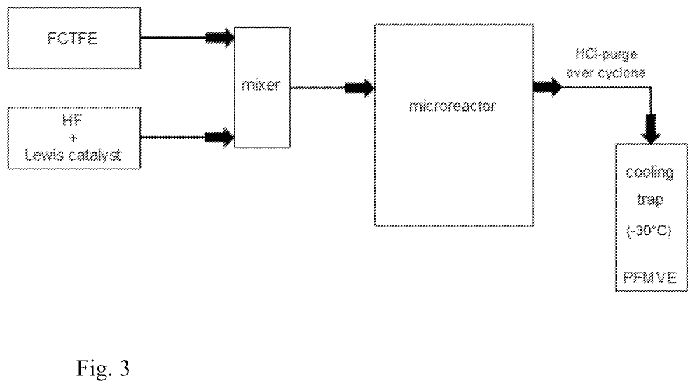

Optionally, any intermediate in the process for manufacturing of perfluoro(methylvinylether) (PFMVE) and/or of 2-fluoro-1,2-dichloro-trifluoro-methoxyethylene (FCTFE) compound according to the invention may be isolated and/or purified, and then such isolated and/or purified may be further processed, as desired. For example, the compound 2-fluoro-1,2-dichloro-trifluoro-methoxyethylene (FCTFE), also known as 1,2-dichloro-1-fluoro-2-(trifluoromethoxy)-ethene (CAS number: 94720-91-9), which is a suitable intermediate in the manufacture of perfluoro(methyl vinyl ether) (PFMVE), may be isolated and/or purified. For example, the compound 2-fluoro-1,2-dichloro-trifluoro-methoxyethylene (FCTFE) is prepared in a first microreactor sequence by addition (A) and elimination (B) reaction (see, for example, FIG. 1, microreactor 1 [SiC] and microreactor 2 [Ni]), is optionally isolated and/or purified, and then the compound 2-fluoro-1,2-dichloro-trifluoro-methoxyethylene (FCTFE) is transferred into another microreactor (see, for example, FIG. 3), to be further reacted with dosed liquid HF (fluorinating agent), especially anhydrous HF (hydrogen fluoride) or water-free HF (hydrogen fluoride), respectively. A Lewis acid is present as a fluorination promoting catalyst, for example, SbF₅, as used for example, in Example 4 or in in Example 6, respectively.

The intermediate compound 2-fluoro-1,2-dichloro-trifluoro-methoxyethylene (FCTFE) produced in the mentioned first microreactor sequence by addition (A) and elimination (B) reaction, optionally may be isolated and/or purified, and then can also constitute the final product in isolated and/or purified form.

Alternatively, (intermediate) compound 2-fluoro-1,2-dichloro-trifluoro-methoxyethylene (FCTFE) produced in a first microreactor sequence by addition (A) and elimination (B) reaction (see, for example, FIG. 1, microreactor 1 [SiC] and microreactor 2 [Ni]), as a crude compound as obtained (e.g., not further purified), is transferred into the mentioned another microreactor (see, for example, FIG. 3), to be further reacted by fluorination with (preferably) anhydrous HF (hydrogen fluoride) to yield the final target compound perfluoro(methyl vinyl ether) (PFMVE). Again, a Lewis acid is present as a fluorination promoting catalyst, for example, SbF₅, as used for example, in Example 4 or in in Example 6, respectively.

In a further variant of the present invention, see for example, Example 4 or in in Example 6, respectively, and reaction Scheme 2, the final target compound perfluoro(methylvinylether) (PFMVE) can also be prepared out of the (intermediate) compound 2-fluoro-1,2-dichloro-trifluoro-methoxyethylene (FCTFE), and described herein above in more detail. Preferably, the reaction can be performed in a continuous manner.

Fluorination catalyst, with Lewis acid properties:

The processes step (C) of the invention employs a fluorination catalyst. Fluorination is a chemical reaction that involves the addition of one or more fluorine (F) atoms to a compound or material. Fluorination is well known to those skilled in the art, as well as suitable fluorination catalysts involved in these reactions.

Fluorination catalysts are well known to those skilled in the field, and preferably in context of the invention, based on Sb, As, Bi, Al, Zn, Fe, Mg, Cr, Ru, Sn, Ti, Co, Ni, preferably on the basis of Sb.

The invention in this regard also relates to a process, for example, wherein the fluorination catalyst is preferably on the basis of Sb, and more preferably is selected from the group consisting of Sb fluorination catalysts providing the active species H₂F⁺SbF₆⁻.

The invention relates to a process, for example, wherein the fluorination catalyst is antimony pentafluoride, preferably wherein the catalyst is antimony pentafluoride (SbF₅) and is prepared in an autoclave by reaction of SbCl₅ with HF, more preferably consisting of SbF₅ in HF which forms the active species H₂F⁺SbF₆⁻, prior to fluorination reaction step (C) in the process according to any one of embodiments of the invention.

Liquid Phase Fluorination/Addition with HF in Presence of Lewis Acid:

The fluorination/addition process with HF in the presence of a Lewis acid catalyst according to the invention is performed in the liquid phase, by the addition reaction of HF (hydrogen fluoride) and elimination of HCl (hydrogen chloride), both in liquid phase, and wherein the addition reaction of HF and elimination of HCl is induced by a Lewis acid.

Preferably, the fluorination reaction with HF (hydrogen fluoride) according to the present invention is performed in that liquid HF (the fluorinating agent), especially anhydrous HF (hydrogen fluoride) or water-free HF (hydrogen fluoride), respectively, is dosed into the reaction under Lewis acid catalysis.

In the fluorination/addition process with HF in the presence of a Lewis acid catalyst according to the invention, the Lewis acid is a metal halogenide, preferable a metal halogenide selected from the group consisting of SbCl₅/SbF₅, TiCl₄/TiF₄, SnCl₄/SnF₄, FeCl₃/FeF₃, ZnCl₂/ZnF₂, or is preferably fluorination promoting catalyst on the basis of Sb, with Lewis acid properties, and more preferably is selected from the group consisting of Sb fluorination catalysts providing the active species H₂F⁺SbF₆⁻ as mentioned above.

Microreactor Process:

The invention also may pertain to a process for manufacturing of perfluoro(methylvinylether) (PFMVE), and/or of 2-fluoro-1,2-dichloro-trifluoro-methoxyethylene (FCTFE), also known as 1,2-dichloro-1-fluoro-2-(trifluoromethoxy)-ethene (CAS number: 94720-91-9), which is a suitable intermediate in the manufacture of perfluoro(methylvinylether) (PFMVE), wherein the process is a continuous process, preferably wherein the continuous process is carried out in a microreactor.

The invention may employ more than a single microreactor, .i.e., the invention may employ two, three, four, five or more microreactors, for either extending the capacity or residence time, for example, to up to ten microreactors in parallel or four microreactors in series. If more than a single microreactor is employed, then the plurality of microreactors can be arranged either sequentially or in parallel, and if three or more microreactors are employed, these may be arranged sequentially, in parallel or both.

The invention is also very advantageous, in to embodiments wherein the process for manufacturing of perfluoro(methyl vinyl ether) (PFMVE) and/or of 2-fluoro-1,2-dichloro-trifluoro-methoxyethylene (FCTFE) according to the invention optionally is performed in a continuous flow reactor system, or preferably in a microreactor system.

In an preferred embodiment the invention relates to a process for manufacturing of perfluoro(methyl vinyl ether) (PFMVE) and/or of 2-fluoro-1,2-dichloro-trifluoro-methoxyethylene (FCTFE), wherein in at least one reaction step is carried out as a continuous processes, wherein the continuous process is performed in at least one continuous flow reactor with upper lateral dimensions of about ≤5 mm, or of about ≤4 mm,

preferably in at least one microreactor; more preferably wherein of the said at least one reaction step is a continuous process in at least one microreactor under one or more of the following conditions:
flow rate: of from about 10 ml/h up to about 400 l/h;
temperature: of from about 30° C. up to about 150° C.;
pressure: of from about 4 bar up to about 50 bar;
residence time: of from about 1 second, preferably from about 1 minute, up to about 60 minutes.

In another preferred embodiment the invention relates to such a process of preparing a compound according to the invention, wherein at least one of the said continuous flow reactors, preferably at least one of the microreactors, independently is a SiC-continuous flow reactor, preferably independently is a SiC-microreactor.

The Continuous Flow Reactors and Microreactors:

In addition to the above, according to one aspect of the invention, also a plant engineering invention is provided, as used in the process invention and described herein, pertaining to the optional, and in some embodiments of the process invention, the process even preferred implementation in microreactors.

As to the term “microreactor”: A “microreactor” or “microstructured reactor” or “microchannel reactor”, in one embodiment of the invention, is a device in which chemical reactions take place in a confinement with typical lateral dimensions of about ≤1 mm; an example of a typical form of such confinement are microchannels. Generally, in the context of the invention, the term “microreactor”: A “microreactor” or “microstructured reactor” or “microchannel reactor”, denotes a device in which chemical reactions take place in a confinement with typical lateral dimensions of about ≤5 mm.

Microreactors are studied in the field of micro process engineering, together with other devices (such as micro heat exchangers) in which physical processes occur. The microreactor is usually a continuous flow reactor (contrast with/to a batch reactor). Microreactors offer many advantages over conventional scale reactors, including vast improvements in energy efficiency, reaction speed and yield, safety, reliability, scalability, on-site/on-demand production, and a much finer degree of process control.

Microreactors are used in “flow chemistry” to perform chemical reactions.

In flow chemistry, wherein often microreactors are used, a chemical reaction is run in a continuously flowing stream rather than in batch production. Batch production is a technique used in manufacturing, in which the object in question is created stage by stage over a series of workstations, and different batches of products are made. Together with job production (one-off production) and mass production (flow production or continuous production) it is one of the three main production methods. In contrast, in flow chemistry the chemical reaction is run in a continuously flowing stream, wherein pumps move fluid into a tube, and where tubes join one another, the fluids contact one another. If these fluids are reactive, a reaction takes place. Flow chemistry is a well-established technique for use at a large scale when manufacturing large quantities of a given material. However, the term has only been coined recently for its application on a laboratory scale.

Continuous flow reactors, e.g. such as used as microreactor, are typically tube like and manufactured from non-reactive materials, such known in the prior art and depending on the specific purpose and nature of possibly aggressive agents and/or reactants. Mixing methods include diffusion alone, e.g. if the diameter of the reactor is narrow, e.g. <1 mm, such as in microreactors, and static mixers. Continuous flow reactors allow good control over reaction conditions including heat transfer, time and mixing. The residence time of the reagents in the reactor, i.e. the amount of time that the reaction is heated or cooled, is calculated from the volume of the reactor and the flow rate through it: Residence time=Reactor Volume/Flow Rate. Therefore, to achieve a longer residence time, reagents can be pumped more slowly, just a larger volume reactor can be used and/or even several microreactors can be placed in series, optionally just having some cylinders in between for increasing residence time if necessary for completion of reaction steps. In this later case, cyclones after each microreactor help to let formed HCl to escape and to positively influence the reaction performance. Production rates can vary from milliliters per minute to liters per hour.

Some examples of flow reactors are spinning disk reactors (Colin Ramshaw); spinning tube reactors; multi-cell flow reactors; oscillatory flow reactors; microreactors; hex reactors; and aspirator reactors. In an aspirator reactor a pump propels one reagent, which causes a reactant to be sucked in. Also to be mentioned are plug flow reactors and tubular flow reactors.

In the present invention, in one embodiment it is particularly preferred to employ a microreactor.

In the use and processes according to the invention in a preferred embodiment the invention is using a microreactor. But it is to be noted in a more general embodiment of the invention, apart from the said preferred embodiment of the invention that is using a microreactor, any other, e.g. preferentially pipe-like, continuous flow reactor with upper lateral dimensions of up to about 1 cm, and as defined herein, can be employed. Thus, such a continuous flow reactor preferably with upper lateral dimensions of up to about ≤5 mm, or of about ≤4 mm, refers to a preferred embodiment of the invention, e.g. preferably to a microreactor. Continuously operated series of STRs is another option, but less preferred than using a microreactor.

In the before said embodiments of the invention, the minimal lateral dimensions of the, e.g. preferentially pipe-like, continuous flow reactor can be about >5 mm; but is usually not exceeding about 1 cm. Thus, the lateral dimensions of the, e.g. preferentially pipe-like, continuous flow reactor can be in the range of from about >5 mm up to about 1 cm, and can be of any value therein between. For example, the lateral dimensions of the, e.g. preferentially pipe-like, continuous flow reactor can be about 5.1 mm, about 5.5 mm, about 6 mm, about 6.5 mm, about 7 mm, about 7.5 mm, about 8 mm, about 8.5 mm, about 9 mm, about 9.5 mm, and about 10 mm, or can be can be of any value intermediate between the said values.

In the before said embodiments of the invention using a microreactor preferentially the minimal lateral dimensions of the microreactor can be at least about 0.25 mm, and preferably at least about 0.5 mm; but the maximum lateral dimensions of the microreactor does not exceed about ≤5 mm. Thus, the lateral dimensions of the, e.g. preferential microreactor can be in the range of from about 0.25 mm up to about 5 mm, and preferably from about 0.5 mm up to about 5 mm, and can be of any value therein between. For example, the lateral dimensions of the preferential microreactor can be about 0.25 mm, about 0.3 mm, about 0.35 mm, about 0.4 mm, about 0.45 mm, and about 5 mm, or can be can be of any value intermediate between the said values.

As stated here before in the embodiments of the invention in its broadest meaning is employing, preferentially pipe-like, continuous flow reactor with upper lateral dimensions of up to about 1 cm. Such continuous flow reactor, for example is a plug flow reactor (PFR).

The plug flow reactor (PFR), sometimes called continuous tubular reactor, CTR, or piston flow reactors, is a reactor used to perform and describe chemical reactions in continuous, flowing systems of cylindrical geometry. The PFR reactor model is used to predict the behavior of chemical reactors of such design, so that key reactor variables, such as the dimensions of the reactor, can be estimated.

Fluid going through a PFR may be modeled as flowing through the reactor as a series of infinitely thin coherent “plugs”, each with a uniform composition, traveling in the axial direction of the reactor, with each plug having a different composition from the ones before and after it. The key assumption is that as a plug flows through a PFR, the fluid is perfectly mixed in the radial direction (i.e. in the lateral direction) but not in the axial direction (forwards or backwards).

Accordingly, the terms used herein to define the reactor type used in the context of the invention such like “continuous flow reactor”, “plug flow reactor”, “tubular reactor”, “continuous flow reactor system”, “plug flow reactor system”, “tubular reactor system”, “continuous flow system”, “plug flow system”, “tubular system” are synonymous to each other and interchangeably by each other.

The reactor or system may be arranged as a multitude of tubes, which may be, for example, linear, looped, meandering, circled, coiled, or combinations thereof. If coiled, for example, then the reactor or system is also called “coiled reactor” or “coiled system”.

In the radial direction, i.e. in the lateral direction, such reactor or system may have an inner diameter or an inner cross-section dimension (i.e. radial dimension or lateral dimension, respectively) of up to about 1 cm. Thus, in an embodiment the lateral dimension of the reactor or system may be in the range of from about 0.25 mm up to about 1 cm, preferably of from about 0.5 mm up to about 1 cm, and more preferably of from about 1 mm up to about 1 cm.

In further embodiments the lateral dimension of the reactor or system may be in the range of from about >5 mm to about 1 cm, or of from about 5.1 mm to about 1 cm.

If the lateral dimension at maximum of up to about ≤5 mm, or of up to about ≤4 mm, then the reactor is called “microreactor”. Thus, in still further microreactor embodiments the lateral dimension of the reactor or system may be in the range of from about 0.25 mm up to about ≤5 mm, preferably of from about 0.5 mm up to about ≤5 mm, and more preferably of from about 1 mm up to about ≤5 mm; or the lateral dimension of the reactor or system may be in the range of from about 0.25 mm up to about ≤4 mm, preferably of from about 0.5 mm up to about ≤4 mm, and more preferably of from about 1 mm up to about ≤4 mm.

In an alternative embodiment of the invention, it is also optionally desired to employ another continuous flow reactor than a microreactor, preferably if, for example, the (halogenation promoting, e.g. the halogenation or preferably the halogenation) catalyst composition used in the halogenation or fluorination tends to get viscous during reaction or is viscous already as a said catalyst as such. In such case, a continuous flow reactor, i.e. a device in which chemical reactions take place in a confinement with lower lateral dimensions of greater than that indicated above for a microreactor, i.e. of greater than about 1 mm, but wherein the upper lateral dimensions are about ≤4 mm. Accordingly, in this alternative embodiment of the invention, employing a continuous flow reactor, the term “continuous flow reactor” preferably denotes a device in which chemical reactions take place in a confinement with typical lateral dimensions of from about ≥1 mm up to about ≤4 mm. In such an embodiment of the invention it is particularly preferred to employ as a continuous flow reactor a plug flow reactor and/or a tubular flow reactor, with the said lateral dimensions. Also, in such an embodiment of the invention, as compared to the embodiment employing a microreactor, it is particularly preferred to employ higher flow rates in the continuous flow reactor, preferably in the plug flow reactor and/or a tubular flow reactor, with the said lateral dimensions. For example, such higher flow rates, are up to about 2 times higher, up to about 3 times higher, up to about 4 times higher, up to about 5 times higher, up to about 6 times higher, up to about 7 times higher, or any intermediate flow rate of from about ≥1 up to about ≤7 times higher, of from about ≥1 up to about ≤6 times higher, of from about ≥1 up to about ≤5 times higher, of from about ≥1 up to about ≤4 times higher, of from about ≥1 up to about ≤3 times higher, or of from about ≥1 up to about ≤2 times higher, each as compared to the typical flow rates indicated herein for a microreactor. Preferably, the said continuous flow reactor, more preferably the the plug flow reactor and/or a tubular flow reactor, employed in this embodiment of the invention is configured with the construction materials as defined herein for the microreactors. For example, such construction materials are silicon carbide (SiC) and/or are alloys such as a highly corrosion resistant nickel-chromium-molybdenum-tungsten alloy, e.g. Hastelloy®, as described herein for the microreactors.

A very particular advantage of the present invention employing a microreactor, or a continuous flow reactor with the before said lateral dimensions, the number of separating steps can be reduced and simplified, and may be devoid of time and energy consuming, e.g. intermediate, distillation steps. Especially, it is a particular advantage of the present invention employing a microreactor, or a continuous flow reactor with the before said lateral dimensions, that for separating simply phase separation methods can be employed, and the non-consumed reaction components may be recycled into the process, or otherwise be used as a product itself, as applicable or desired.

In addition to the preferred embodiments of the present invention using a microreactor according to the invention, in addition or alternatively to using a microreactor, it is also possible to employ a plug flow reactor or a tubular flow reactor, respectively.

Plug flow reactor or tubular flow reactor, respectively, and their operation conditions, are well known to those skilled in the field.

Although the use of a continuous flow reactor with upper lateral dimensions of about ≤5 mm, or of about ≤4 mm, respectively, and in particular of a microreactor, is particularly preferred in the present invention, depending on the circumstances, it could be imagined that somebody dispenses with an microreactor, then of course with yield losses and higher residence time, higher temperature, and instead takes a plug flow reactor or turbulent flow reactor, respectively. However, this could have a potential advantage, taking note of the mentioned possibly disadvantageous yield losses, namely the advantage that the probability of possible blockages (tar particle formation by non-ideal driving style) could be reduced because the diameters of the tubes or channels of a plug flow reactor are greater than those of a microreactor.

The possibly allegeable disadvantage of this variant using a plug flow reactor or a tubular flow reactor, however, may also be seen only as subjective point of view, but on the other hand under certain process constraints in a region or at a production facility may still be appropriate, and loss of yields be considered of less importance or even being acceptable in view of other advantages or avoidance of constraints.

In the following, the invention is more particularly described in the context of using a microreactor. Preferentially, a microreactor used according to the invention is a ceramic continuous flow reactor, more preferably an SiC (silicon carbide) continuous flow reactor, and can be used for material production at a multi-to scale. Within integrated heat exchangers and SiC materials of construction, it gives optimal control of challenging flow chemistry application. The compact, modular construction of the flow production reactor enables, advantageously for: long term flexibility towards different process types; access to a range of production volumes (5 to 400 l/h); intensified chemical production where space is limited; unrivalled chemical compatibility and thermal control.

Ceramic (SiC) microreactors, are e.g. advantageously diffusion bonded 3M SiC reactors, especially braze and metal free, provide for excellent heat and mass transfer, superior chemical compatibility, of FDA certified materials of construction, or of other drug regulatory authority (e.g. EMA) certified materials of construction. Silicon carbide (SiC), also known as carborundum, is a containing silicon and carbon, and is well known to those skilled in the art. For example, synthetic SiC powder is been mass-produced and processed for many technical applications.

For example, in the embodiments of the invention the objects are achieved by a method in which at least one reaction step takes place in a microreactor. Particularly, in preferred embodiments of the invention the objects are achieved by a method in which at least one reaction step takes place in a microreactor that is comprising or is made of SiC (“SiC-microreactor”), or in a microreactor that is comprising or is made of an alloy, e.g. such as Hastelloy C, as it is each defined herein after in more detail.

Preferred Hastelloy C4 nickel alloys are already described further above. See, for example, Table 1.

Thus, without being limited to, for example, in an embodiment of the invention the microreactor suitable for, preferably for industrial, production an “SiC-microreactor” that is comprising or is made of SiC (silicon carbide; e.g. SiC as offered by Dow Corning as Type GlSiC or by Chemtrix MR555 Plantrix), e.g. providing a production capacity of from about 5 up to about 400 kg per hour; or without being limited to, for example, in another embodiment of the invention the microreactor suitable for industrial production is comprising or is made of Hastelloy C, as offered by Ehrfeld. Such microreactors are particularly suitable for the, preferably industrial, production of fluorinated products according to the invention.

In order to meet both the mechanical and chemical demands placed on production scale flow reactors, Plantrix modules are fabricated from 3M™ SiC (Grade C). Produced using the patented 3M (EP 1 637 271 B1 and foreign patents) diffusion bonding technology, the resulting monolithic reactors are hermetically sealed and are free from welding lines/joints and brazing agents. More technical information on the Chemtrix MR555 Plantrix can be found in the brochure “CHEMTRIX—Scalable Flow Chemistry—Technical Information Plantrix® MR555 Series, published by Chemtrix BV in 2017, which technical information is incorporated herein by reference in its entirety.

Apart from the before said example, in other embodiments of the invention, in general SiC from other manufactures, and as known to the skilled person, of course can be employed in the present invention.

Accordingly, in the present invention as microreactor also the Protrix® of by Chemtrix can be used. Protrix® is a modular, continuous flow reactor fabricated from 3M® silicon carbide, offering superior chemical resistance and heat transfer. In order to meet both the mechanical and chemical demands placed on flow reactors, Protrix® modules are fabricated from 3M® SiC (Grade C). Produced using the patented 3M (EP 1 637 271 B1 and foreign patents) diffusion bonding technology, the resulting monolithic reactors are hermetically sealed and are free from welding lines/joints and brazing agents. This fabrication technique is a production method that gives solid SiC reactors (thermal expansion coefficient=4.1×10⁻⁶K⁻¹).

Designed for flow rates ranging from 0.2 to 20 ml/min and pressures up to 25 bar, Protrix® allows the user to develop continuous flow processes at the lab-scale, later transitioning to Plantrix® MR555 (×340 scale factor) for material production. The Protrix® reactor is a unique flow reactor with the following advantages: diffusion bonded 3M® SiC modules with integrated heat exchangers that offer unrivaled thermal control and superior chemical resistance; safe employment of extreme reaction conditions on a g scale in a standard fume hood; efficient, flexible production in terms of number of reagent inputs, capacity or reaction time. The general specifications for the Protrix® flow reactors are summarized as follows; possible reaction types are, e.g. A+B→P1+Q (or C)→P, wherein the terms “A”, “B” and “C” represent educts, “P” and “P1” products, and “Q” quencher; throughput (ml/min) of from about 0.2 up to about 20; channel dimensions (mm) of 1×1 (pre-heat and mixer zone), 1.4×1.4 (residence channel); reagent feeds of 1 to 3; module dimensions (width×height) (mm) of 110×260; frame dimensions (width×height×length) (mm) approximately 400×300×250; number of modules/frame is one (minimum) up to four (max.). More technical information on the ChemtrixProtrix® reactor can be found in the brochure “CHEMTRIX—Scalable Flow Chemistry—Technical Information Protrix®, published by Chemtrix BV in 2017, which technical information is incorporated herein by reference in its entirety.

The Dow Corning as Type GlSiC microreactor, which is scalable for industrial production, and as well suitable for process development and small production can be characterized in terms of dimensions as follows: typical reactor size (length×width×height) of 88 cm×38 cm×72 cm; typical fluidic module size of 188 mm×162 mm. The features of the Dow Corning as Type GlSiC microreactor can be summarized as follows: outstanding mixing and heat exchange: patented HEART design; small internal volume; high residence time; highly flexible and multipurpose; high chemical durability which makes it suitable for high pH compounds and especially hydrofluoric acid; hybrid glass/SiC solution for construction material; seamless scale-up with other advanced-flow reactors. Typical specifications of the Dow Corning as Type GlSiC microreactor are as follows: flow rate of from about 30 ml/min up to about 200 ml/min; operating temperature in the range of from about −60° C. up to about 200° C., operating pressure up to about 18 barg (“barg” is a unit of gauge pressure, i.e. pressure in bars above ambient or atmospheric pressure); materials used are silicon carbide, PFA (perfluoroalkoxy alkanes), perfluoroelastomer; fluidic module of 10 ml internal volume; options: regulatory authority certifications, e.g. FDA or EMA, respectively. The reactor configuration of Dow Corning as Type GlSiC microreactor is characterized as multipurpose and configuration can be customized. Injection points may be added anywhere on the said reactor.

Hastelloy® C is an alloy represented by the formula NiCr21Mo14W, alternatively also known as “alloy 22” or “Hastelloy® C-22. The said alloy is well known as a highly corrosion resistant nickel-chromium-molybdenum-tungsten alloy and has excellent resistance to oxidizing reducing and mixed acids. The said alloy is used in flue gas desulphurization plants, in the chemical industry, environmental protection systems, waste incineration plants, sewage plants. Apart from the before said example, in other embodiments of the invention, in general nickel-chromium-molybdenum-tungsten alloy from other manufactures, and as known to the skilled person, of course can be employed in the present invention. A typical chemical composition (all in weight-%) of such nickel-chromium-molybdenum-tungsten alloy is, each percentage based on the total alloy composition as 100%: Ni (nickel) as the main component (balance) of at least about 51.0%, e.g. in a range of from about 51.0% to about 63.0%; Cr (chromium) in a range of from about 20.0 to about 22.5%, Mo (molybdenum) in a range of from about 12.5 to about 14.5%, W (tungsten or wolfram, respectively) in a range of from about 2.5 to about 3.5%; and Fe (iron) in an amount of up to about 6.0%, e.g. in a range of from about 1.0% to about 6.0%, preferably in a range of from about 1.5% to about 6.0%, more preferably in a range of from about 2.0% to about 6.0%. Optionally, the percentage based on the total alloy composition as 100%, Co (cobalt) can be present in the alloy in an amount of up to about 2.5%, e.g. in a range of from about 0.1% to about 2.5%. Optionally, the percentage based on the total alloy composition as 100%, V (vanadium) can be present in the alloy in an amount of up to about 0.35%, e.g. in a range of from about 0.1% to about 0,35%. Also, the percentage based on the total alloy composition as 100%, optionally low amounts (i.e. ≤0.1%) of other element traces, e.g. independently of C (carbon), Si (silicon), Mn (manganese), P (phosphor), and/or S (sulfur). In such case of low amounts (i.e. ≤0.1%) of other elements, the said elements e.g. of C (carbon), Si (silicon), Mn (manganese), P (phosphor), and/or S (sulfur), the percentage based on the total alloy composition as 100%, each independently can be present in an amount of up to about 0.1%, e.g. each independently in a range of from about 0.01 to about 0.1%, preferably each independently in an amount of up to about 0.08%, e.g. each independently in a range of from about 0.01 to about 0.08%. For example, said elements e.g. of C (carbon), Si (silicon), Mn (manganese), P (phosphor), and/or S (sulfur), the percentage based on the total alloy composition as 100%, each independently can be present in an amount of, each value as an about value: C≤0.01%, Si≤0.08%, Mn≤0.05%, P≤0.015%, S≤0.02%. Normally, no traceable amounts of any of the following elements are found in the alloy compositions indicated above: Nb (niobium), Ti (titanium), Al (aluminum), Cu (copper), N (nitrogen), and Ce (cerium).

Hastelloy® C-276 alloy was the first wrought, nickel-chromium-molybdenum material to alleviate concerns over welding (by virtue of extremely low carbon and silicon contents). As such, it was widely accepted in the chemical process and associated industries, and now has a 50-year-old track record of proven performance in a vast number of corrosive chemicals. Like other nickel alloys, it is ductile, easy to form and weld, and possesses exceptional resistance to stress corrosion cracking in chloride-bearing solutions (a form of degradation to which the austenitic stainless steels are prone). With its high chromium and molybdenum contents, it is able to withstand both oxidizing and non-oxidizing acids, and exhibits outstanding resistance to pitting and crevice attack in the presence of chlorides and other halides. The nominal composition in weight-% is, based on the total composition as 100%: Ni (nickel) 57% (balance); Co (cobalt) 2.5% (max.); Cr (chromium) 16%; Mo (molybdenum) 16%; Fe (iron) 5%; W (tungsten or wolfram, respectively) 4%; further components in lower amounts can be Mn (manganese) up to 1% (max.); V (vanadium) up to 0.35% (max.); Si (silicon) up to 0.08% (max.); C (carbon) 0.01 (max.); Cu (copper) up to 0.5% (max.).

In another embodiments of the invention, without being limited to, for example, the microreactor suitable for the said production, preferably for the said industrial production, is an SiC-microreactor that is comprising or is made only of SiC as the construction material (silicon carbide; e.g. SiC as offered by Dow Corning as Type GlSiC or by Chemtrix MR555 Plantrix), e.g. providing a production capacity of from about 5 up to about 400 kg per hour.

It is of course possible according to the invention to use one or more microreactors, preferably one or more SiC-microreactors, in the production, preferably in the industrial production, of the fluorinated products according to the invention. If more than one microreactor, preferably more than one SiC-microreactor, are used in the production, preferably in the industrial production, of the fluorinated products according to the invention, then these microreactors, preferably these SiC-microreactors, can be used in parallel and/or subsequent arrangements. For example, two, three, four, or more microreactors, preferably two, three, four, or more SiC-microreactors, can be used in parallel and/or subsequent arrangements.

For laboratory search, e.g. on applicable reaction and/or upscaling conditions, without being limited to, for example, as a microreactor the reactor type Plantrix of the company Chemtrix is suitable. Sometimes, if gaskets of a microreactor are made out of other material than HDPTFE, leakage might occur quite soon after short time of operation because of some swelling, so HDPTFE gaskets secure long operating time of microreactor and involved other equipment parts like settler and distillation columns.

For example, an industrial flow reactor (“IFR”, e.g. Plantrix® MR555) comprises of SiC modules (e.g. 3M® SiC) housed within a (non-wetted) stainless steel frame, through which connection of feed lines and service media are made using standard Swagelok fittings. The process fluids are heated or cooled within the modules using integrated heat exchangers, when used in conjunction with a service medium (thermal fluid or steam), and reacted in zig-zag or double zig-zag, meso-channel structures that are designed to give plug flow and have a high heat exchange capacity. A basic IFR (e.g. Plantrix® MR555) system comprises of one SiC module (e.g. 3M® SiC), a mixer (“MRX”) that affords access to A+B→P type reactions. Increasing the number of modules leads to increased reaction times and/or system productivity. The addition of a quench Q/C module extends reaction types to A+B→P1+Q (or C)→P and a blanking plate gives two temperature zones. Herein the terms “A”, “B” and “C” represent educts, “P” and “P1” products, and “Q” quencher.

Typical dimensions of an industrial flow reactor (“IFR”, e.g. Plantrix® MR555) are, for example: channel dimensions in (mm) of 4×4 (“MRX”, mixer) and 5×5 (MRH-I/MRH-II; “MRH” denotes residence module); module dimensions (width×height) of 200 mm×555 mm; frame dimensions (width×height) of 322 mm×811 mm. A typical throughput of an industrial flow reactor (“IFR”, e.g. Plantrix® MR555) is, for example, in the range of from about 50 l/h to about 400 l/h. in addition, depending on fluid properties and process conditions used, the throughput of an industrial flow reactor (“IFR”, e.g. Plantrix® MR555), for example, can also be >400 l/h. The residence modules can be placed in series in order to deliver the required reaction volume or productivity. The number of modules that can be placed in series depends on the fluid properties and targeted flow rate.

Typical operating or process conditions of an industrial flow reactor (“IFR”, e.g. Plantrix® MR555) are, for example: temperature range of from about −30° C. to about 200° C.; temperature difference (service—process)<70° C.; reagent feeds of 1 to 3; maximum operating pressure (service fluid) of about 5 bar at a temperature of about 200° C.; maximum operating pressure (process fluid) of about 25 bar at a temperature of about ≤200° C.

The following examples are intended to further illustrate the invention without limiting its scope.

### EXAMPLES

The following examples are intended to further illustrate the invention without limiting its scope.

### Example 1

Preparation of CF₃OF (not Inventive).

CF₃OF was prepared out of CO with excess F₂ according to JACS 70 (1948) 3986.

Alternatively, CF₃OF can also be prepared by a two-step procedure over COF₂ as intermediate, which procedure is described by in EP 1801091 (2006; Solvay Solexis).

### Example 2

Reaction of CF₃OF with Trifluoroethylene in Two Microreactors.

The reactions in this example were performed in a two microreactor system as shown in FIG. 1.

### Example 2a

Two 27 ml micoreactors (first one made out of SiC, second one made of Ni) were installed in series, the first microreactor was kept at room temperature (ambient temperature; about 25° C.) by cooling, and the second microreactor was heated to 100° C., pressure was adjusted to 4 bar abs. by using a pressure valve installed at the gas exit at the cyclone.

There was a cooler installed after the second microreactor to immediately cool down the reaction mixture to 0° C. (cooler not shown in the FIG. 1) where the desired product PFMVE is a gas and formed HF is in liquid state already. Also, after the second microreactor and after the cooler, the partially liquid reaction mixture was fed into a cyclone with said pressure valve at gas exit (cyclone also not shown in the FIG. 1). The liquid phase material of the cyclone (HF) moved into a storage tank for a re-use. The gas phase of the cyclone consisted mainly out of PFMVE (together with only some traces HF) and moves over a Swagelok hand valve (for further expanding to about normal pressure, e.g., at 1 atm) into the cooling trap by means of a deep pipe (a stainless steel cylinder equipped with deep pipe and a gas outlet); the cooling trap was kept at about −30° C.

Before starting the reaction, the system is continuously floated with a He (helium) inert gas purge which purge was rapidly reduced once the feeding of raw materials has started and purge was stopped completely after reaching constant feed of the raw materials into the reactor. A fast reduction of inert gas feed (purge) is essential as inert gas reduces sharply the heat exchange efficiency in both reactors.

Into this reactor installation, floated with a He (helium) inert, CF₃OF was fed out of a gas cylinder (out of gaseous phase) over a Bronkhorst mass flow controller together with gaseous trifluoroethylene out of another cylinder with 150 g (1.83 mol/h) and over a Bronkhorst mass flow controller in a ratio of 1.05:1.0.

Final distillation of the collected PFMVE was done in a pressure column made out of Hastelloy C4 (nickel alloy), at 5 bar abs. yielding 96% of PFMVE (99.9% GC-purity) based on trifluoroethylene starting material.

### Example 2b

In another trial, the cooler and the cyclone, both were put out of order, and all the gaseous material leaving the second microreactor over the pressure valve at the cyclone was expanded to about normal pressure, e.g., at 1 atm abs.; and all product material was condensed in the cooling trap at −30° C. Afterwards, the PFMVE/HF mixture was slowly neutralized with NEt₃ in a Hastelloy vessel at 4 bar abs. for quenching the HF, a second lower phase was formed which contained the product PFMVE in 93% yield.

### Example 3

Preparation of FCTFE Out of Trichloroethylene and CF₃OF in Two Microreactors.

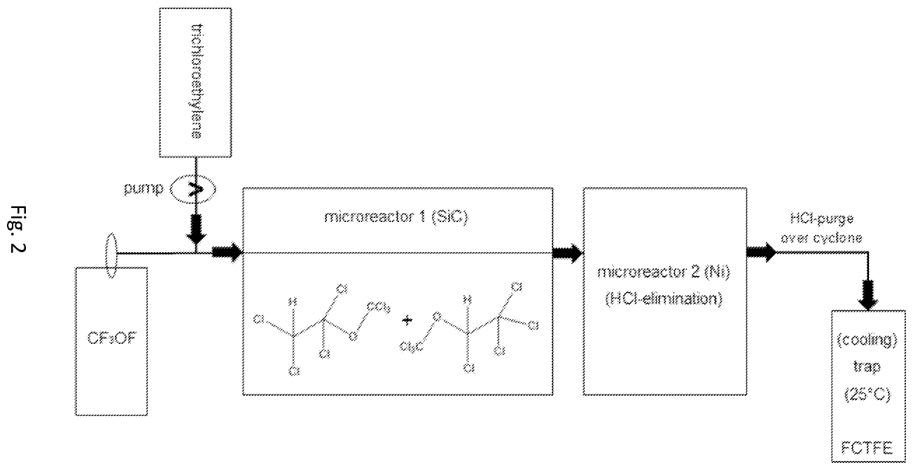

The reactions in this example were performed in a two microreactor system as shown in FIG. 2.

Two 27 ml micoreactors (first one made out of SiC, second one made out of Ni) were installed in series, the first microreactor was kept at 25° C. (room temperature (ambient temperature; about 25° C.) by cooling, the second microreactor was heated to 100° C. The pressure was adjusted to 4 bar abs. by using a pressure valve installed at the gas exit at the cyclone. See also Example 2.

Before starting the reaction, the system is continuously floated with a He (helium) inert gas purge which purge was rapidly reduced once the feeding of raw materials has started and purge was stopped completely after reaching constant feed of raw materials into the reactor. A fast reduction of inert gas feed once dosage has started is essential as inert gas reduces sharply the heat exchange efficiency in both reactors.

Into this reactor installation CF₃OF was fed out of a gas cylinder (out of gaseous phase) over a Bronkhorst mass flow controller together with liquid trichloroethylene (TRI) out of a storage tank in a ratio of 1.05:1.0. The TRI feed was set to 120 g/h (0.91 mol/h).

As in example 2, there was a cooler installed after the second microreactor to cool down the reaction mixture to 0° C. (cooler not shown in the FIG. 2). After the second microreactor and after the cooler, the reaction mixture was fed into a cyclone (cyclone also not shown in the FIG. 2), the liquid phase in the cyclone flows moved over a Swagelok hand valve (further expanding to about normal pressure, e.g., at 1 atm) into the (cooling) trap (25° C.) by means of a deep pipe (a stainless steel cylinder equipped with a deep pipe and a gas outlet). The gas phase in the cyclone (with the HCl) moved into an efficient scrubber.

Most of HCl was already purged over the cyclone into a scrubber and FCTFE is collected in the above said cooling trap kept at 25° C. with some traces of dissolved HCl. Final distillation of FCTFE was done in a stainless steel column, at 1 bar abs., and yielded 89% FCTFE based on trichloroethylene starting material (with 99.2% GC-purity) at transition temperature of 98° C.

### Example 4

Conversion of FCTFE to PFMVE by Fluorination with HF (in Batch) and SbF₅ as Lewis Acid.

For final fluorination of FCTFE to PFMVE, the FCTFE obtained in Example was taken out of the cooling trap of Example 3.

In a 250 ml Roth autoclave with an inner liner out of HDPTFE (HDPTFE=High Density TetraFluoroEthylene), and with a pressure valve installed at the gas exit adjusted to 8 bar abs., a quantity of 40 g (0.2 mol) FCTFE was slowly fed over a deep pipe into the autoclave which contained 7.9 g (0.04 mol) SbF₅ in 100 g HF free of water, a slight exothermic activity could be observed. The SbF₅/HF mixture was prepared in advance by just slowly feeding SbCl₅ with a piston pump into the autoclave which was preloaded with HF, at room temperature (ambient temperature) (about 25° C.), and while keeping the pressure at 3 bar abs. by some HCl gas purge during this pre-fluorination procedure. After finishing the FCTFE-feed, then the autoclave was heated in an oil bath to 80° C. for 1 h, some HCl could be observed leaving the autoclave over the pressure valve kept at 8 bar abs. during all the time. After cooling down the content (autoclave emptied over deep pipe) was slowly fed into another HDPTFE coated pressure cylinder having a volume of 500 ml and been kept at 5 bar abs., the said pressure cylinder contained 50 ml of ice water to finally get rid of the excess HF; towards the end of emptying the autoclave, N₂ pressure was applied at the gas phase inlet of the autoclave to get the complete content out. An organic phase (lower phase) was formed in the pressure cylinder which contained 60% (GC) of PFMVE and 32% (GC) of dichloro-difluoroethyl-trifluoromethyl ether identified by GC-MS (50 m CP-SIL column from Angilent), together with 8% (GC) of not converted FCTFE. GC-samples were injected as gas phase samples.

### Example 5

Conversion of FCTFE to PFMVE by Fluorination with HF (in Batch) and SnCl₄ as Lewis Acid.

Example 4 was repeated, but instead of SbCl₅ in Example 4, SnCl₄ (same amount) was used as Lewis acid. The FCTFE conversion was 29%, after work up the organic phase contained only 3% PFMVE and mainly dichlorodifluoroethyl-trifluoromethyl ether identified by GC-MS (50 m CP-SIL column from Angilent), besides the starting material.

### Examples 6, 7 and 8

### Example 6, 7 and 8: Conversion of FCTFE to PFMVE by Fluorination with HF (in Continuous Manner) and Lewis Acids

Reference is made to the reaction scheme displayed in FIG. 3 showing a continuous synthesis in microreactor. Various Lewis acids were used in the Examples 6, 7 and 8 as shown herein after.

### Example 6

In example 6, SbF₅ was used as Lewis acid and fed as mixture with HF out of a stainless steel cylinder. FCTFE as obtained in Example 3 was fed together with that HF/catalyst mixture into a 27 ml SiC microreactor from Chemtrix which was heated to 75° C. (pressure=8 bar abs.). A quantity of 150 g (0.75 mol) FCTFE was reacted over 1 h with an excess of 40 g (2.0 mol) HF with 3.16 g (0.02 mol) dissolved SbF₅. There is a cooler installed after the microreactor (not shown in FIG. 3) also out of SiC to cool down the reaction mixture to 0° C. which then is moved into a cyclone (also not shown in FIG. 3). The gas phase of the cyclone (mainly HCl) was fed into an efficient scrubber, the liquid phase was expanded over a Swagelok hand valve to 1 bar abs. into a cooling trap which was cooled with dry ice/methanol mixture (−30° C.) to isolate PFMVE as Work up of the content of the cooling trap as in Example 4 into ice water gave an organic phase which contained 97 GC-% PFMVE and only traces of not converted FCTFE.

### Example 7

In example 7, pre-fluorinated TiCl₄ was used as Lewis acid. The procedure of Example 6 was repeated. The conversion was only 47%, the organic phase mainly contained dichlorodifluoroethyl-trifluoromethyl ether confirmed by GC-MS and only traces of PFMVE.

### Example 8

In example 8, pre-fluorinated SnCl₄ was used as Lewis acid. The procedure of Example 6 was repeated. The conversion was 56%, the organic phase contained dichlorodifluoroethyl-trifluoromethyl ether confirmed by GC-MS and 10 GC-% of PFMVE.

### Example 9

Preparation of FCTFE in a Counter-Current Reactor Out of Trichloroethylene and CF₃OF.

The reactions in this example were performed in a two microreactor system as shown in FIG. 4.

Apparatus: A column made out of Hastelloy C4 (nickel alloy) with a length of 30 cm and with 10 mm Hastelloy fillings (Pall-ring type from company Raschig) and a diameter of 5 cm was used according to the drawing below. The liquid reservoir with a filling level measurement had a volume of 21 also made out of Hastelloy. The pump was a centrifugal pump from company Schmitt. A pressure valve on top of the tower was installed to regulate the pressure. A heat exchanger for heating and cooling was installed into the loop as drawn. For the thermolysis step (second step), the gas stream (FCTFE/HCl) leaving the apparatus over a pressure valve installed at the top was connected to a cooling trap kept at 0° C. which is not shown in the FIG. 4.

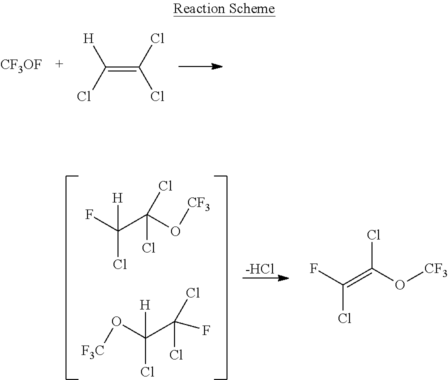

The reservoir was filled with 1000 g (7.6 mol) trichloroethylene, the pump for the loop was started (flow of about 1500 l/h) while cooling to 0° C., the pressure valve was set to 2 bar abs. Once the circulating fluid has reached 0° C., CF₃OF was fed out of a gas cylinder over a Bronkhorst mass flow meter with 405.6 (3.9 mol) per hour into the tower so that the reaction temperature was kept below 5° C. After 2 h a quantity of 811.2 g (7.8 mol) CF₃OF was completely fed into the system. After further 15 min of looping, a slight N₂-inert gas stream (100 l/h) was added at the inlet which before was used for the CF₃OF feed. Now the pressure was kept at 2 bar abs., the cooling trap was put into operation for collecting the FCTFE (and some dissolved HCl), the mixture was slowly heated to 100° C. At 70° C., some HCl-evolution started which became much stronger at 100° C. As the volume in the reservoir was shrinking to 100 ml (as the cooling trap was collecting the product), the thermolysis was stopped and the reservoir was filled again with another 1000 g of Trichloroethylene to restart the procedure with the CF₃OF feeding step for producing more material.

The material collected in the cooling trap finally was neutralized by washing with ice water and dried over Na₂SO₄. After filtration, a GC analysis showed a 98.3% purity for obtained FCTFE (82% yield) so it could be used without any further purification for the fluorination step.

What is claimed is:

1. A process for the manufacture of PFMVE (perfluoro(methyl vinyl ether)) having the formula (I), 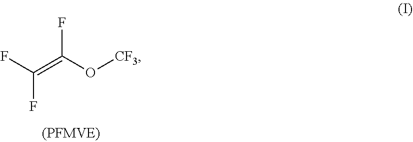
characterized in that a trihalomethyl hypohalogenite of formula (III) and a trihaloethylene of formula (IV) are reacted with each other,
CX₃—O—X (III),
wherein, in formula (III), X represents F (fluorine atom) or Cl (chlorine atom), 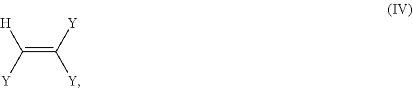
wherein, in formula (IV), Y represents F (fluorine atom) or Cl (chlorine atom);
and wherein the process comprises the steps of performing:
(A) in a first step in a first reactor, with the proviso that if the trihaloethylene of formula (IV) is a gaseous starting material then the first reactor is not a loop reactor, preferably wherein the first reactor is a microreactor, an addition reaction, wherein the trihalomethyl hypohalogenite of formula (III) is added to the trihaloethylene of formula (IV) and the addition reaction is performed at a temperature in the range of about 0° C. to about 35° C. to form an addition product (A-P); and subsequently, with or without isolating the (liquid) addition product (A-P),
(B) in a second step in said first reactor if the first reactor is a loop reactor, or in a second a reactor, which is a microreactor, in liquid phase an elimination reaction, wherein HY (hydrogen halogenide) is eliminated from the addition product (A-P) and the elimination reaction is performed at a temperature in the range of about 80° C. to about 120° C. to yield a trihalomethoxytrihaloethylene compound of formula (V), 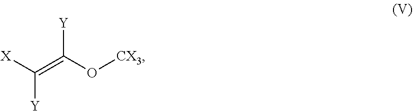
wherein in formula (V), X represents F (fluorine atom) or Cl (chlorine atom), Y represents F (fluorine atom) or Cl (chlorine atom);
and with the provisos (i) and (ii) that
(i) if X and Y are the same in each of the compounds of formulae (III) to (V), and each of X and Y represents F (fluorine atom), directly the compound of formula (I), PFMVE (perfluoro(methyl vinyl ether)), is obtained; and
(ii) if X and Y are different from each other in that either X represents F (fluorine atom) and Y represents Cl (chlorine atom), or X represents Cl (chlorine atom) and Y represents F (fluorine atom),
(C) then in a third reactor, preferably wherein the third reactor is microreactor, the trihalomethoxytrihaloethylene compound of formula (V) is subjected to a fluorination reaction in liquid phase, wherein the trihalomethoxytrihaloethylene compound of formula (V) is fluorinated with HF (hydrogen fluoride) in the presence of at least one Lewis acid catalyst, and at a temperature in the range of about 50° C. to about 100° C., in order to replace the Cl (chlorine atom) substituents contained in the compound of formula (V) by F (fluorine atom), by addition of HF and elimination of HCl (hydrogen chloride), and thereby to obtain the compound of formula (I), PFMVE (perfluoro(methyl vinyl ether)).

2. The process according to claim 1, for the manufacture of PFMVE (perfluoro(methyl vinyl ether)) having the formula (I), characterized in that X in the trihalomethyl hypohalogenite of formula (III) represents F (fluorine atom).

3. The process according to claim 1, for the manufacture of PFMVE (perfluoro(methyl vinyl ether)) having the formula (I), characterized in that Y in the trihaloethylene of formula (IV) represents F (fluorine atom).

4. The process according to claim 1, for the manufacture of PFMVE (perfluoro(methyl vinyl ether)) having the formula (I), characterized in that X in the trihalomethyl hypohalogenite of formula (III) and Y in the trihaloethylene of formula (IV) both represent F (fluorine atom).

5. The process according to claim 1, for the manufacture of PFMVE (perfluoro(methyl vinyl ether)) having the formula (I), 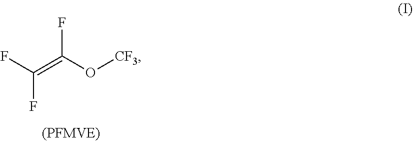
characterized in that a trifluoromethyl hypofluorite of formula (IIIa) and a trifluoroethylene of formula (IVa) are reacted with each other, 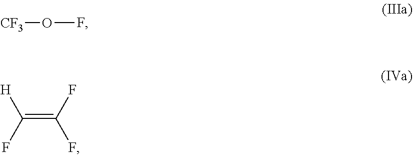 [SMILES: FOC(F)(F)F ; FC=C(F)F]
and wherein the process comprises the steps of performing:
(A) in a first step in a first reactor, with the proviso that the first reactor is not a loop reactor, preferably wherein the first reactor is microreactor, an addition reaction, wherein the trifluoromethyl hypofluorite of formula (IIIa) is added to the trifluoroethylene of formula (IVa) and the addition reaction is performed at a temperature in the range of about 0° C. to about 35° C. to form an addition product (A-Paa); and subsequently, with or without isolating the (liquid) addition product (A-P),
(B) in a second step in a second reactor, preferably microreactor, in liquid phase an elimination reaction, wherein HF (hydrogen fluoride) is eliminated from the addition product (A-Paa) and the elimination reaction is performed at a temperature in the range of about 80° C. to about 120° C. to obtain the compound of formula (I), PFMVE (perfluoro(methyl vinyl ether)).

6. The process according to claim 1, for the manufacture of PFMVE (perfluoro(methyl vinyl ether)) having the formula (I), 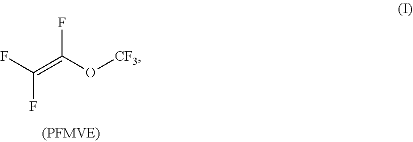
characterized in that a trifluoromethyl hypofluorite of formula (IIIa) and a trichloroethylene of formula (IV) are reacted with each other, 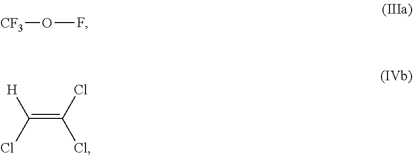 [SMILES: FOC(F)(F)F ; ClC=C(Cl)Cl]
and wherein the process comprises the steps of performing:
(A) in a first step in a first reactor, preferably in a loop reactor or in a micro reactor, more preferably in a microreactor, an addition reaction, wherein the trifluoromethyl hypofluorite of formula (IIIa) is added to the trichloroethylene of formula (IVb) and the addition reaction is performed at a temperature in the range of about 0° C. to about 35° C. to form an addition product (A-Pab); and subsequently, with or without isolating the (liquid) addition product (A-P),
(B) in a second step in said first reactor if the first reactor is a loop reactor, or in a second a reactor, which is a microreactor, in liquid phase an elimination reaction, wherein HY (hydrogen halogenide) is eliminated from the addition product (A-Pab) and the elimination reaction is performed at a temperature in the range of about 80° C. to about 120° C. to yield a compound of formula (II) (2-fluoro-1,2-dichloro-trifluoro-methoxyethylene), 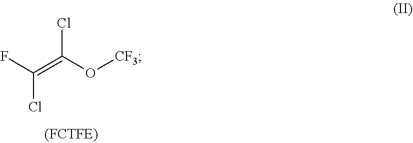
and
(C) then in a third reactor the compound of formula (II) (2-fluoro-1,2-dichloro-trifluoro-methoxyethylene) is subjected to a fluorination reaction in liquid phase, wherein the compound of formula (II) is fluorinated with HF (hydrogen fluoride) in the presence of at least one Lewis acid catalyst, and at a temperature in the range of about 50° C. to about 100° C., in order to replace the Cl (chlorine) atoms contained in the compound of formula (II) by F (fluorine) atoms, by addition of HF and elimination of HCl (hydrogen chloride), and thereby to obtain the compound of formula (I), PFMVE (perfluoro(methyl vinyl ether)).

7. A process for the manufacture of PFMVE (perfluoro(methyl vinyl ether)) having the formula (I), 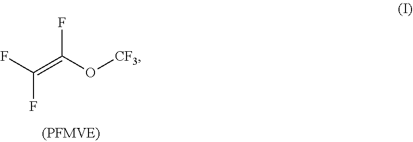
characterized in that the process comprises performing a step (C):
(C) wherein in a reactor, preferably wherein the reactor is microreactor, a compound of formula (II) (2-fluoro-1,2-dichloro-trifluoro-methoxyethylene), 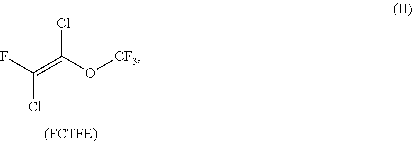
is subjected to a fluorination reaction in liquid phase, wherein the compound of formula (II) (2-fluoro-1,2-dichloro-trifluoro-methoxyethylene) is fluorinated with HF (hydrogen fluoride) in the presence of at least one Lewis acid catalyst, and at a temperature in the range of about 50° C. to about 100° C., in order to replace the Cl (chlorine) atoms contained in the compound of formula (II) by F (fluorine) atoms, by addition of HF and elimination of HCl (hydrogen chloride), and thereby to obtain the compound of formula (I), PFMVE (perfluoro(methyl vinyl ether)).

8. The process according to claim 1 for the manufacture of PFMVE (perfluoro(methyl vinyl ether)) having the formula (I), characterized in that in step (C) the fluorination reaction is performed at a temperature in the range of about 50° C. to about 100° C., preferably at a temperature in the range of about 60° C. to about 100° C., more preferably at a temperature in the range of about 60° C. to about 90° C., even more preferably at a temperature in the range of about 70° C. to about 90° C. (or a temperature of about 80° C.±10° C.), still more preferably at a temperature in the range of about 70° C. to about 80° C. (or a temperature of about 100° C.±5° C.), or at a temperature of about 75° C. (e.g., at a temperature of about 75° C.±4° C., or 75° C.±3° C., or 75° C.±2° C., or 75° C.±1° C.).

9. The process according to claim 1, for the manufacture of PFMVE (perfluoro(methyl vinyl ether)) having the formula (I), characterized in that in step (C) the fluorination reaction is performed in a continuous manner, preferably in a continuous manner in a microreactor.

10. The process according to claim 1 for the manufacture of PFMVE (perfluoro(methyl vinyl ether)) having the formula (I), characterized in that in step (C) the fluorination reaction is performed in the presence of a Lewis acid catalyst selected from the group consisting of SnCl₄ (tin tetrachloride), TiCl₄ (titanium tetrachloride), and SbF₅ (antimony pentafluoride).

11. The process according to claim 10 for the manufacture of PFMVE (perfluoro(methyl vinyl ether)) having the formula (I), characterized in that in step (C) the fluorination reaction is performed in the presence of the Lewis acid catalyst SbF₅ (antimony pentafluoride).

12. A process for the manufacture of FCTFE (2-fluoro-1,2-dichloro-trifluoro-methoxyethylene) having the formula (II), 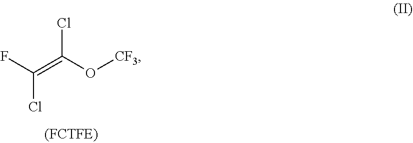
characterized in that a trifluoromethyl hypofluorite of formula (IIIa) and a trichloroethylene of formula (IVb) are reacted with each other, 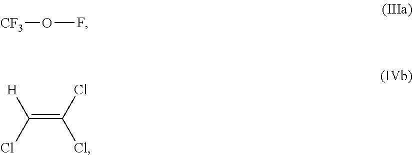 [SMILES: FOC(F)(F)F ; ClC=C(Cl)Cl]
and wherein the process comprises the steps of performing:
(A) in a first step in a first reactor, preferably in a loop reactor or in a micro reactor, more preferably in a microreactor, an addition reaction, wherein the trifluoromethyl hypofluorite of formula (IIIa) is added to the trichloroethylene of formula (IVb) and the addition reaction is performed at a temperature in the range of about 0° C. to about 35° C. to form an addition product (A-Pab); and subsequently, with or without isolating the (liquid) addition product (A-P),
(B) in a second step in said first reactor if the first reactor is a loop reactor, or in a second a reactor, which is a microreactor, in liquid phase an elimination reaction, wherein HCl (hydrogen chloride) is eliminated from the addition product (A-Pab) and the elimination reaction is performed at a temperature in the range of about 80° C. to about 120° C. to obtain the compound of formula (II), FCTFE (2-fluoro-1,2-dichloro-trifluoro-methoxyethylene).

13. The process according to claim 12 for the manufacture of FCTFE (2-fluoro-1,2-dichloro-trifluoro-methoxyethylene) having the formula (II), characterized in that in step (A) in the first step in the first reactor the addition reaction is performed at a temperature in the range of about 15° C. to about 35° C. (or a temperature of about 25° C.±10° C.), preferably at a temperature in the range of about 20° C. to about 30° C. (or a temperature of about 25° C.±5° C.), more preferably at ambient (or room) temperature (or a temperature of about 20° C. to about 25° C.).

14. The process according to claim 12 for the manufacture of FCTFE (2-fluoro-1,2-dichloro-trifluoro-methoxyethylene) having the formula (II), characterized in that in step (B) in the second step in said first reactor if the first reactor is a loop reactor, or in a second reactor, which is a microreactor, the elimination reaction is performed at a temperature in the range of about 90° C. to about 110° C. (or a temperature of about 100° C.±10° C.), preferably at a temperature in the range of about 95° C. to about 105° C. (or a temperature of about 100° C.±5° C.), or at a temperature of about 100° C. (e.g., at a temperature of about 100° C. 4° C., or 100° C. 3° C., or 100° C. 2° C., or 100° C. 1° C.).

15. The process according to claim 12 for the manufacture of FCTFE (2-fluoro-1,2-dichloro-trifluoro-methoxyethylene) having the formula (II), characterized in that prior to starting any of the process steps (A), (B), and (C) (if applicable), one or more of the reactors used, preferably each and any of the reactors used, are purged with an inert gas, preferably with He (helium) as the inert gas.

16. The process according to claim 12 for the manufacture of FCTFE (2-fluoro-1,2-dichloro-trifluoro-methoxyethylene) having the formula (II), characterized in that in step (A) the in the first reactor the addition reaction is performed in a SiC-reactor.

17. The process according to claim 12 for the manufacture of FCTFE (2-fluoro-1,2-dichloro-trifluoro-methoxyethylene) having the formula (II), characterized in that in step (B) in the second reactor the elimination reaction is performed in a nickel-reactor (Ni-reactor) or in a reactor with an inner surface with high nickel-content (Ni-content).

18. The process according to claim 12 for the manufacture of FCTFE (2-fluoro-1,2-dichloro-trifluoro-methoxyethylene) having the formula (II), characterized in that that in step (A) the addition reaction is performed in a continuous manner, preferably in a continuous manner in a microreactor.

19. The process according to claim 12 for the manufacture of FCTFE (2-fluoro-1,2-dichloro-trifluoro-methoxyethylene) having the formula (II), characterized in that in step (B) the elimination reaction is performed in a continuous manner, preferably in a continuous manner in a microreactor.

20. The process according to claim 19 for the manufacture of FCTFE (2-fluoro-1,2-dichloro-trifluoro-methoxyethylene) having the formula (II), characterized in that, independently the reaction in at least one reaction step of (A), (B), and (C) (if applicable), is carried as a continuous processes, wherein the continuous process in the at least one reaction step of (A), (B), and (C) (if applicable), is performed in at least one continuous flow reactor with upper lateral dimensions of about ≤5 mm, or of about ≤4 mm, preferably wherein at least one of the continuous flow reactor is a microreactor.

21. The process according to claim 20 for the manufacture of FCTFE (2-fluoro-1,2-dichloro-trifluoro-methoxyethylene) having the formula (II), characterized in that the reaction is carried out in at least one reaction step of (A), (B), and (C) (if applicable), as a continuous processes, wherein the continuous process is performed in at least one continuous flow reactor with upper lateral dimensions of about ≤5 mm, or of about ≤4 mm, preferably in at least one microreactor;
more preferably wherein of the said steps of (A), (B), and (C), at least the step (C) of a fluorination reaction is a continuous process in at least one microreactor under one or more of the following conditions:
flow rate: of from about 10 ml/h up to about 400 l/h;
temperature: ranging of from about −20° C. or of from about −10° C. or of from about 0° C. or of from about 10° C., or of from about 20° C. or of from about 30° C., respectively, each ranging to up to about 150° C.;
pressure: of from about 1 bar (1 atm abs.) up to about 50 bar; preferably of from about 1 bar (1 atm abs.) up to about 20 bar, more preferably at about 1 bar (1 atm abs.) up to about 5 bar; most preferably at about 1 bar (1 atm abs.) up to about 4 bar; in an example the pressure is about 3 bar;
residence time: of from about 1 second, preferably from about 1 minute, up to about 60 minutes.

22. The process according to claim 12 for the manufacture of FCTFE (2-fluoro-1,2-dichloro-trifluoro-methoxyethylene) having the formula (II), characterized in that, independently, the product yielding from step (A), the product resulting from step (B) and/or the product yielding from step (C) (if applicable) are subjected to distillation.
